(19) United States
(12) Patent Application Publication
(10) Pub. No.: US 2021/0108253 A1
(43) Pub. Date: Apr. 15, 2021

(54) QUANTIFICATION OF NGS DNA BY ADAPTER SEQUENCE

(71) Applicant: Life Technologies Corporation, Carlsbad, CA (US)

(72) Inventors: Andrew DIX, Eugene, OR (US); Jeffrey Monette, Eugene, OR (US)

(21) Appl. No.: 16/497,615

(22) PCT Filed: Mar. 29, 2018
(86) PCT No.: PCT/US2018/025055

§ 371 Date: Sep. 25, 2019

Related U.S. Application Data
(60) Provisional application No. 62/478,825, filed on Mar. 30, 2017.

(51) Int. Cl. C12Q 1/6818; C12Q 1/6876

(52) U.S. Cl. CPC C12Q 1/6818; C12Q 1/6876

(57) ABSTRACT

The present disclosure is directed to methods and kits for detection and/or quantification of nucleic acids, such as double stranded DNA that is used in next-generation sequencing (NGS) applications. Nucleic acid-based probes, such as peptide nucleic acid (PNA) oligomers, molecular beacons, DNA flares and locked nucleic acid (LNA) oligomers, are also provided for use in the methods and kits of the present disclosure.

### CROSS-REFERENCE

This application is a National Stage 371 from PCT/US2018/025055 filed Mar. 29, 2018, which claims the benefit under 35 U.S.C. § 119(e) of U.S. Provisional Application No. 62/478,825, filed Mar. 30, 2017. The entire contents of the aforementioned applications are incorporated by reference herein.

### SEQUENCE LISTING

The instant application contains a Sequence Listing which has been submitted electronically in ASCII format and is hereby incorporated by reference in its entirety. Said ASCII copy, created on Jun. 26, 2018, is named LT01235PCT_SL.txt and is 16,518 bytes in size.

### FIELD OF THE INVENTION

This disclosure generally relates to the field of nucleic acid quantification, and more particularly, to methods of nucleic acid quantification for next-generation sequencing (NGS) applications.

### BRIEF SUMMARY OF THE INVENTION

Provided herein are methods for quantifying nucleic acids for use in next-generation sequencing (NGS) applications, as well as nucleic acid-based probes and kits for use in such methods.

In certain embodiments of the present disclosure, a method for detecting a nucleic acid in a sample is provided, the method comprising:
a) combining a nucleic acid probe with the sample to prepare a probe-nucleic acid mixture;
b) incubating the probe-nucleic acid mixture for a sufficient amount of time for the nucleic acid probe to hybridize to the nucleic acid in the sample to form a probe-nucleic acid complex;
c) illuminating the probe-nucleic acid complex with an appropriate wavelength of light to generate a detectable optical response; and
d) detecting the detectable optical response thereby detecting the nucleic acid.

In certain embodiments of the present disclosure, a method for quantifying a nucleic acid in a sample is provided, the method comprising:
a) combining a nucleic acid probe with the sample to prepare a probe-nucleic acid mixture;
b) incubating the probe-nucleic acid mixture for a sufficient amount of time for the nucleic acid probe to hybridize to the nucleic acid in the sample to form a probe-nucleic acid complex;
c) illuminating the probe-nucleic acid complex with an appropriate wavelength of light to generate a detectable optical response;
d) detecting the detectable optical response; and
e) comparing the detectable optical response with a known quantity of nucleic acid thereby quantifying the nucleic acid in the sample.

In certain embodiments of the present disclosure, a method of detecting a target nucleic acid in a sample is provided, the method comprising:
a) providing a sample containing a target nucleic acid comprising at least one sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence; and
c) incubating the target-probe mixture for a sufficient amount of time to allow the adapter-specific probe to hybridize to the target nucleic acid to form a probe-target complex.

In certain embodiments, the method further comprises:
d) illuminating the probe-target complex with an appropriate wavelength of light to generate a detectable optical response; and
e) detecting the detectable optical response thereby detecting the target nucleic acid in the sample.

In certain preferred embodiments, the adapter-specific probe is complementary to the 3′-end region or the 5′-end region of the sequencing by synthesis adapter sequence. In certain preferred embodiments, the detectable optical response is proportional to the amount of target nucleic acid present in the sample.

In certain embodiments of the present disclosure, a method of detecting a target nucleic acid in a sample is provided, the method comprising:
a) providing a sample containing a target-probe mixture, wherein the target-probe mixture comprises i) a target nucleic acid comprising at least one sequencing by synthesis adapter sequence, and ii) an adapter-specific probe comprising a nucleic acid sequence that is complementary to the sequencing by synthesis adapter sequence;
b) illuminating the target-probe mixture with an appropriate wavelength of light to generate a detectable optical response; and
c) detecting the detectable optical response thereby detecting the target nucleic acid in the sample.

In certain additional embodiments of the present disclosure, a method of detecting a target nucleic acid in a sample is provided, the method comprising:
a) providing a sample containing one or more target-probe mixtures, wherein the one or more target-probe mixtures comprises i) a target nucleic acid comprising at least one sequencing by synthesis adapter sequence, and ii) one or more adapter-specific probe comprising a nucleic acid sequence that is complementary to the sequencing by synthesis adapter sequence;
b) illuminating the one or more target-probe mixture with an appropriate wavelength of light to generate a detectable optical response; and
c) detecting the detectable optical response thereby detecting the target nucleic acid in the sample.

In certain embodiments, the sample contains two target-probe mixtures. In certain embodiments, the sample contains three target-probe mixtures. In certain preferred embodiments, the adapter-specific probe is complementary to the 3′-end or the 5′-end region of the sequencing by synthesis adapter sequence.

In certain embodiments of the present disclosure, a method of detecting a target nucleic acid in a sample is provided, the method comprising:
a) providing a sample containing a target nucleic acid comprising at least one sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence; and
d) incubating the first target-probe mixture and the second target-probe mixture for a sufficient amount of time to allow the first adapter-specific probe and the second adapter-specific probe to hybridize to the target nucleic acid to form a first probe-target complex and a second probe-target complex;
wherein the first adapter-specific probe and the second adapter-specific probe are detectably distinct.

In certain embodiments, the method further comprises:
e) illuminating the first probe-target complex and the second probe-target complex with an appropriate wavelength of light to generate a first detectable optical response and a second detectable optical response; and
f) detecting the first detectable optical response and the second detectable optical response thereby detecting the target nucleic acid in the sample.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the detectable optical response is proportional to the amount of target nucleic acid present in the sample.

In certain embodiments of the present disclosure, a method of quantifying a target nucleic acid in a sample is provided, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence; and
c) incubating the target-probe mixture for a sufficient amount of time to allow the adapter-specific probe to hybridize to the target nucleic acid to form a probe-target complex.

In certain embodiments, the method further comprises:
d) illuminating the probe-target complex with an appropriate wavelength of light to generate a detectable optical response;
e) detecting the detectable optical response; and
f) comparing the detectable optical response with a known quantity of nucleic acid thereby quantifying the target nucleic acid in the sample.

In certain preferred embodiments, the adapter-specific probe is complementary to the 3′-end or the 5′-end of the sequencing by synthesis adapter sequence. In certain preferred embodiments, the detectable optical response is proportional to the amount of target nucleic acid present in the sample.

In certain embodiments of the present disclosure, a method of quantifying a target nucleic acid in a sample is provided, the method comprising:
a) providing a sample containing a target nucleic acid comprising at least one sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence; and
d) incubating the first target-probe mixture and the second target probe mixture for a sufficient amount of time to allow the first adapter-specific probe and the second adapter-specific probe to hybridize to the target nucleic acid to form a first probe-target complex and a second probe-target complex;
wherein the first adapter-specific probe and the second adapter-specific probe are detectably distinct.

In certain embodiments, the method further comprises:
e) illuminating the first probe-target complex and the second probe-target complex with an appropriate wavelength of light to generate a first detectable optical response and a second detectable optical response; and
f) detecting the first detectable optical response and the second detectable optical response thereby detecting the target nucleic acid in the sample;
g) comparing the first detectable optical response and the second detectable optical response with a known quantity of nucleic acid thereby quantifying the target nucleic acid in the sample.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the detectable optical response is proportional to the amount of target nucleic acid present in the sample.

In certain embodiments, a method of quantifying a target nucleic acid in a sample is provided, the method comprising:
a) providing a sample containing a target-probe mixture, wherein the target-probe mixture comprises i) a target nucleic acid comprising a sequencing by synthesis adapter sequence, and ii) an adapter-specific probe comprising a nucleic acid sequence that is complementary to the sequencing by synthesis adapter sequence;
b) illuminating the target-probe mixture with an appropriate wavelength of light to generate a detectable optical response;
c) detecting the detectable optical response; and
d) comparing the detectable optical response with a known quantity of nucleic acid thereby quantifying the target nucleic acid in the sample.

In certain embodiments, a method of quantifying a target nucleic acid in a sample is provided, the method comprising:
a) providing a sample containing one or more target-probe mixtures, wherein the one or more target-probe mixtures comprises i) a target nucleic acid comprising at least one sequencing by synthesis adapter sequence, and ii) one or more adapter-specific probes comprising a nucleic acid sequence that is complementary to the sequencing by synthesis adapter sequence;
b) illuminating the one or more target-probe mixture with an appropriate wavelength of light to generate a detectable optical response; and
c) detecting the detectable optical response thereby detecting the target nucleic acid in the sample.

In certain embodiments, the sample contains two target-probe mixtures. In certain embodiments, the sample contains three target-probe mixtures. In certain preferred embodiments, the adapter-specific probe is complementary to the 3′-end or the 5′-end of the sequencing by synthesis adapter sequence.

In certain embodiments of the present disclosure, a method of detecting a target nucleic acid in a sample is provided, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence;
c) heating the target-probe mixture for a sufficient amount of time to allow the target nucleic acid to denature;
d) cooling the target-probe mixture for a sufficient amount of time to allow the adapter-specific probe to hybridize to the target nucleic acid to create a probe-target complex;
e) illuminating the probe-target complex with an appropriate wavelength of light to generate a detectable optical response; and
f) detecting the detectable optical response thereby detecting the target nucleic acid in the sample.

In certain preferred embodiments, the adapter-specific probe is complementary to the 3′-end or the 5′-end of the sequencing by synthesis adapter sequence. In certain preferred embodiments, the detectable optical response is proportional to the amount of target nucleic acid present in the sample.

In certain embodiments of the present disclosure, a method of detecting a target nucleic acid in a sample is provided, the method comprising:
a) providing a sample containing a target nucleic acid comprising a sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence;
d) heating the first target-probe mixture and the second target-probe mixture for a sufficient amount of time to allow the target nucleic acid to denature;
e) cooling the first target-probe mixture and the second target-probe mixture for a sufficient amount of time to allow the first adapter-specific probe and the second adapter-specific probe to hybridize to the target nucleic acid to create a first probe-target complex and a second probe-target complex;
f) illuminating the first probe-target complex and the second probe-target complex with an appropriate wavelength of light to generate a first detectable optical response and a second detectable optical response; and
g) detecting the first detectable optical response and the second detectable optical response thereby detecting the target nucleic acid in the sample,
wherein the first adapter-specific probe and the second adapter-specific probe are detectably distinct.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the detectable optical response is proportional to the amount of target nucleic acid present in the sample.

In certain embodiments, a method of quantifying a target nucleic acid in a sample is provided, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence;
c) heating the target-probe mixture for a sufficient amount of time to allow the target nucleic acid to denature;
d) cooling the target-probe mixture for a sufficient amount of time to allow the adapter-specific probe to hybridize to the target nucleic acid to create a probe-target complex;
e) illuminating the probe-target complex with an appropriate wavelength of light to generate a detectable optical response;
f) detecting the detectable optical response; and
g) comparing the detectable optical response with a known quantity of nucleic acid thereby quantifying the target nucleic acid in the sample.

In certain preferred embodiments, the adapter-specific probe is complementary to the 3′-end or the 5′-end of the sequencing by synthesis adapter sequence. In certain preferred embodiments, the detectable optical response is proportional to the amount of target nucleic acid present in the sample.

In certain embodiments of the present disclosure, a method of quantifying a target nucleic acid in a sample is provided, the method comprising:
a) providing a sample containing a target nucleic acid comprising a sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence;
d) heating the first target-probe mixture and the second target-probe mixture for a sufficient amount of time to allow the target nucleic acid to denature;
e) cooling the first target-probe mixture and the second target-probe mixture for a sufficient amount of time to allow the first adapter-specific probe and the second adapter-specific probe to hybridize to the target nucleic acid to create a first probe-target complex and a second target-probe complex;
f) illuminating the first probe-target complex and the second target-probe complex with an appropriate wavelength of light to generate a first detectable optical response and a second detectable optical response;
g) detecting the first detectable optical response and the second detectable optical response; and
h) comparing the first detectable optical response and the second detectable optical response with a known quantity of nucleic acid thereby quantifying the target nucleic acid in the sample,
wherein the first adapter-specific probe and the second adapter-specific probe are detectably distinct.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the detectable optical response is proportional to the amount of target nucleic acid present in the sample.

In certain embodiments of the methods provided herein, the target nucleic acid is double-stranded DNA.

In certain embodiments of the methods provided herein, the adapter-specific probe comprises an oligonucleotide sequence that is complementary to a nucleic acid sequence of the sequencing by synthesis adapter sequence, or a portion thereof, and a fluorescent dye. In certain embodiments of the methods provided herein, the adapter-specific probe further comprises a quencher. In certain embodiments of the methods provided herein, the adapter-specific probe comprises a peptide nucleic acid (PNA) oligomer probe, a molecular beacon probe, a DNA flare probe or a locked nucleic acid (LNA) probe pair.

In certain embodiments, the adapter-specific probe comprises a peptide nucleic acid (PNA) oligomer comprising a fluorescent dye attached to the N-terminus of the PNA oligomer, a quencher attached to the C-terminus of the PNA oligomer, and a series of nucleotides that are complementary to a nucleic acid sequence of the sequencing by synthesis adapter. In certain embodiments, the PNA oligomer comprises 8-12 nucleotides that are complementary to a nucleic acid sequence of the sequencing by synthesis adapter. In certain embodiments, the adapter-specific probe comprises a molecular beacon comprising a fluorescent dye attached to the 5′-end of the molecular beacon, a quencher attached to the 3′-end of the molecular beacon, and 18 nucleotides in the loop portion that are complementary to a nucleic acid sequence of the sequencing by synthesis adapter. In other certain embodiments, the adapter-specific probe comprises a DNA flare comprising a fluorescent dye covalently attached to one or more nucleotides, and 28 nucleotides that are complementary to a nucleic acid sequence of the sequencing by synthesis adapter. In yet other embodiments, the adapter-specific probe comprises a locked nucleic acid (LNA) probe pair comprising: a first LNA probe comprising a fluorescent dye attached to the 3′-end or the 5′-end of the first probe, and a series of nucleotides that are complementary to a nucleic acid sequence of the sequencing by synthesis adapter; and a second LNA probe comprising a fluorescence quencher attached to either the 3′-end or the 5′-end of the second probe and a series of nucleotides that are complementary to the nucleic acid sequence of the first LNA probe. In certain embodiments, the first LNA probe comprises 8-12 nucleotides that are complementary to a nucleic acid sequence of the sequencing by synthesis adapter. In certain embodiments, the fluorescent dye and/or quencher are directly attached to one or more nucleotides of the adapter-specific probe. In certain embodiments, the fluorescent dye and/or quencher are covalently attached to one or more nucleotides of the adapter-specific probe.

In certain embodiments of the methods provided herein, the methods further comprise adding a primer-dimer detection probe to the target-probe mixture, wherein the primer-dimer detection probe has a detectable optical response that is distinguishable from the detectable optical response of the adapter-specific probe.

In certain embodiments, the fluorescent dye is selected from the group chosen from a pyrene, a xanthene, a cyanine, an indole, a benzofuran, a coumarin, or a borapolyazaindacene. In certain embodiments, the quencher is selected from the group chosen from a BLACK HOLE QUENCHER® dye (Biosearch Technologies Inc., Petaluma, Calif.), an IOWA BLACK® Quencher (Integrated DNA Technologies, Inc., Coralville, Iowa), a QSY® Quencher (Thermo Fisher Scientific, Waltham, Mass.), Dabsyl, Dabcel, a Deep Dark Quencher (Kaneka Eurogentec, S.A., Seraing, Belgium), and an ECLIPSE® Quencher (Glen Research, Sterling, Va.).

In certain embodiments of the methods provided herein, the detecting step is performed by fluorimetry. In certain preferred embodiments, the detecting step is performed on a QUBIT fluorometer (Thermo Fisher Scientific, Waltham, Mass.). In certain embodiments, the sample is in a microfuge tube or a multi-well plate. In certain embodiments, the sequencing by synthesis adapter is a TRUSEQ® Universal Adapter or a TRUSEQ® Indexed Adapter (Illumina, San Diego, Calif.).

In certain embodiments of the present disclosure, an adapter-specific probe is provided, the probe comprising: an oligonucleotide sequence that is complementary to a nucleic acid sequence of a sequencing by synthesis adapter, or a portion thereof, and a fluorescent dye. In certain embodiments, the adapter-specific probe further comprises a quencher. In certain embodiments, the adapter-specific probe comprises a peptide nucleic acid (PNA) oligomer probe, a molecular beacon probe, a DNA flare probe or a locked nucleic acid (LNA) probe pair.

In certain embodiments of the present disclosure, the adapter-specific probe comprises a PNA oligomer probe, wherein the peptide nucleic acid (PNA) oligomer probe comprises a fluorescent dye attached to the N-terminus of the PNA oligomer, a fluorescence quencher attached to the C-terminus of the PNA oligomer, and a series of nucleotides that are complementary to the nucleic acid sequence of the sequencing by synthesis adapter. In certain embodiments, the PNA oligomer comprises 8-12 nucleotides that are complementary to a nucleic acid sequence of the sequencing by synthesis adapter. In certain embodiments, the adapter-specific probe comprises a molecular beacon probe, wherein the molecular beacon probe comprises a fluorescent dye attached to the 5′-end of the molecular beacon, a fluorescence quencher attached to the 3′-end of the molecular beacon, and 18 nucleotides in the loop portion that are complementary to the nucleic acid sequence of the sequencing by synthesis adapter. In certain embodiments, the adapter-specific probe comprises a DNA flare probe, wherein the DNA flare probe comprises a fluorescent dye covalently attached to one or more nucleotides, and 28 nucleotides that are complementary to the nucleic acid sequence of the sequencing by synthesis adapter. In certain embodiments, the adapter-specific probe comprises a locked nucleic acid (LNA) probe pair, wherein the LNA probe pair comprises: a first LNA probe comprising a fluorescent dye attached to the 3′-end or the 5′-end of the first probe, and a series of nucleotides that are complementary to the nucleic acid sequence of the sequencing by synthesis adapter; and a second LNA probe comprising a fluorescence quencher attached to either the 3′-end or the 5′-end of the second probe and a series of nucleotides that are complementary to the nucleic acid sequence of the first LNA probe. In certain embodiments, the first LNA probe comprises 8-12 nucleotides that are complementary to the nucleic acid sequence of the sequencing by synthesis adapter. In certain embodiments, the fluorescent dye is selected from the group chosen from a pyrene, a xanthene, a cyanine, an indole, a benzofuran, a coumarin, or a borapolyazaindacene. In certain embodiments, the quencher is selected from the group chosen from BLACK HOLE QUENCHER® dye (Biosearch Technologies Inc., Petaluma, Calif.), an IOWA BLACK® Quencher (Integrated DNA Technologies, Inc., Coralville, Iowa), a QSY® Quencher (Thermo Fisher Scientific, Waltham, Mass.), Dabsyl, Dabcel, a Deep Dark Quencher (Kaneka Eurogentec, S.A., Seraing, Belgium), and an ECLIPSE® Quencher (Glen Research, Sterling, Va.). In certain embodiments, the fluorescent dye and/or quencher are directly attached to one or more nucleotides of the adapter-specific probe. In certain embodiments, the fluorescent dye and/or quencher are covalently attached to one or more nucleotides of the adapter-specific probe.

In certain embodiments of the present disclosure, a primer-dimer detection probe is provided, the primer-dimer detection probe comprising: an oligonucleotide sequence that is complementary to a nucleic acid sequence of a sequencing by synthesis primer-dimer; and a fluorescent dye. In certain embodiments, the primer-dimer detection probe further comprises a quencher. In certain embodiments, the primer-dimer detection probe comprises a peptide nucleic acid (PNA) oligomer probe, a molecular beacon probe, a DNA flare probe or a locked nucleic acid (LNA) probe pair.

In certain embodiments, the primer-dimer detection probe comprises a PNA oligomer probe, wherein the peptide nucleic acid (PNA) oligomer probe comprises a fluorescent dye attached to the N-terminus of the PNA oligomer, a quencher attached to the C-terminus of the PNA oligomer, and a series of nucleotides that are complementary to the nucleic acid sequence of the sequencing by synthesis primer-dimer. In certain embodiments, the PNA oligomer comprises 8-12 nucleotides that are complementary to the nucleic acid sequence of the sequencing by synthesis primer-dimer. In certain embodiments, the primer-dimer detection probe comprises a molecular beacon probe, wherein the molecular beacon probe comprises a fluorescent dye attached to the 5′-end of the molecular beacon, a quencher attached to the 3′-end of the molecular beacon, and 18 nucleotides in the loop portion that are complementary to the nucleic acid sequence of the sequencing by synthesis primer-dimer. In certain embodiments, the primer-dimer detection probe comprises a DNA flare probe, wherein the DNA flare probe comprises a fluorescent dye covalently attached to one or more nucleotides, and 28 nucleotides that are complementary to the nucleic acid sequence of the sequencing by synthesis primer-dimer. In certain embodiments, the primer-dimer detection probe comprises a locked nucleic acid (LNA) probe pair, wherein the LNA probe pair comprises: a first LNA probe comprising a fluorescent dye attached to the 3′-end or the 5′-end of the first probe and a series of nucleotides that are complementary to the nucleic acid sequence of the sequencing by synthesis primer-dimer; and a second LNA probe comprising a fluorescence quencher attached to either the 3′-end or the 5′-end of the second probe and a series of nucleotides that are complementary the nucleic acid sequence of the first LNA probe. In certain embodiments, the first LNA probe comprises 8-12 nucleotides that are complementary to the nucleic acid sequence of the sequencing by synthesis primer-dimer. In certain embodiments, the fluorescent dye is selected from the group chosen from a pyrene, a xanthene, a cyanine, an indole, a benzofuran, a coumarin, or a borapolyazaindacene, and is detectably distinct from an adapter-specific probe. In certain embodiments, the quencher is selected from the group chosen from BLACK HOLE QUENCHER® dye (Biosearch Technologies Inc., Petaluma, Calif.), an IOWA BLACK® Quencher (Integrated DNA Technologies, Inc., Coralville, Iowa), a QSY® Quencher (Thermo Fisher Scientific, Waltham, Mass.), Dabsyl, Dabcel, a Deep Dark Quencher (Kaneka Eurogentec, S.A., Seraing, Belgium), and an ECLIPSE® Quencher (Glen Research, Sterling, Va.). In certain embodiments, the fluorescent dye and/or quencher are directly attached to one or more nucleotides of the primer-dimer detection probe. In certain embodiments, the fluorescent dye and/or quencher are covalently attached to one or more nucleotides of the primer-dimer detection probe.

In certain embodiments of the present disclosure, a kit for detecting or quantifying nucleic acid is provided, the kit comprising: one or more adapter-specific probe; a buffer; and instructions for detecting or quantifying nucleic acid. In certain embodiments, the kit further comprises a primer-dimer detection probe. In certain embodiments, the kit comprises two adapter-specific probes.

In certain embodiments of the kits provided herein, the adapter-specific probe comprises: an oligonucleotide sequence that is complementary to a nucleic acid sequence of a sequencing by synthesis adapter, or a portion thereof, and a fluorescent dye. In certain embodiments, the adapter-specific probe further comprises a quencher. In certain embodiments, the adapter-specific probe comprises a peptide nucleic acid (PNA) oligomer probe, a molecular beacon probe, a DNA flare probe or a locked nucleic acid (LNA) probe pair. In certain embodiments, the adapter-specific probe comprises a peptide nucleic acid (PNA) oligomer comprising a fluorescent dye attached to the N-terminus of the PNA oligomer, a quencher attached to the C-terminus of the PNA oligomer, and a series of nucleotides that are complementary to the nucleic acid sequence of the sequencing by synthesis adapter. In certain embodiments, the PNA oligomer comprises 8-12 nucleotides that are complementary to a nucleic acid sequence of the sequencing by synthesis adapter. In certain embodiments, the adapter-specific probe comprises a molecular beacon comprising a fluorescent dye attached to the 5′-end of the molecular beacon, a quencher attached to the 3′-end of the molecular beacon, and 18 nucleotides in the loop portion that are complementary to the nucleic acid sequence of the sequencing by synthesis adapter. In certain embodiments, the adapter-specific probe comprises a DNA flare probe comprising a fluorescent dye covalently attached to one or more nucleotides, and 28 nucleotides that are complementary to the nucleic acid sequence of the sequencing by synthesis adapter. In certain embodiments, the adapter-specific probe comprises a locked nucleic acid (LNA) probe pair comprising: a first LNA probe comprising a fluorescent dye attached to the 3′-end or the 5′-end of the first probe and a series of nucleotides that are complementary to the nucleic acid sequence of the sequencing by synthesis adapter; and a second LNA probe comprising a fluorescence quencher attached to either the 3′-end or the 5′-end of the second probe and a series of nucleotides that are complementary to the nucleic acid sequence of the first LNA probe. In certain embodiments, the first LNA probe comprises 8-12 nucleotides that are complementary to the nucleic acid sequence of the sequencing by synthesis adapter. In certain embodiments, the fluorescent dye and/or quencher are directly attached to one or more nucleotides of the adapter-specific probe. In certain embodiments, the fluorescent dye and/or quencher are covalently attached to one or more nucleotides of the adapter-specific probe.

In certain embodiments of the kits provided herein, the primer-dimer detection probe comprises: an oligonucleotide sequence that is complementary to a nucleic acid sequence of a sequencing by synthesis primer-dimer; and a fluorescent dye. In certain embodiments, the primer-dimer detection probe further comprises a fluorescence quencher. In certain embodiments, the primer-dimer detection probe comprises a peptide nucleic acid (PNA) oligomer probe, a molecular beacon probe, a DNA flare probe or a locked nucleic acid (LNA) probe pair. In certain embodiments, the peptide nucleic acid (PNA) oligomer probe comprises a fluorescent dye attached to the N-terminus of the PNA oligomer, a quencher attached to the C-terminus of the PNA oligomer, and a series of nucleotides that are complementary to the nucleic acid sequence of the sequencing by synthesis primer-dimer. In certain embodiments, the PNA oligomer comprises 8-12 nucleotides that are complementary to the nucleic acid sequence of the sequencing by synthesis adapter primer-dimer. In certain embodiments, the molecular beacon probe comprises a fluorescent dye attached to the 5′-end of the molecular beacon, a quencher attached to the 3′-end of the molecular beacon, and 18 nucleotides in the loop portion that are complementary to the nucleic acid sequence of the sequencing by synthesis primer-dimer. In certain embodiments, the DNA flare probe comprises a fluorescent dye covalently attached to one or more nucleotides, and 28 nucleotides that are complementary to the nucleic acid sequence of the sequencing by synthesis primer-dimer. In certain embodiments, the locked nucleic acid (LNA) probe pair comprises: a first LNA probe comprising a fluorescent dye attached to the 3′-end or the 5′-end of the first probe and a series of nucleotides that are complementary to the nucleic acid sequence of the sequencing by synthesis primer-dimer; and a second LNA probe comprising a fluorescence quencher attached to either the 3′-end or the 5′-end of the second probe and a series of nucleotides that are complementary to the nucleic acid sequence of the first LNA probe. In certain embodiments, the first LNA probe comprises 8-12 nucleotides that are complementary to a portion of the sequencing by synthesis primer-dimer. In certain embodiments, the primer-dimer detection probe has a detectable optical response that is distinguishable from the detectable optical response of the adapter-specific probe. In certain embodiments, the fluorescent dye and/or quencher are directly attached to one or more nucleotides of the primer-dimer detection probe. In certain embodiments, the fluorescent dye and/or quencher are covalently attached to one or more nucleotides of the primer-dimer detection probe.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic illustration of adapter modification of DNA using the Illumina sequencing by synthesis platform, wherein the target sequence comprises the sequencing by synthesis adapter sequence which is represented in the figures as a white-colored strand, whereas the genomic DNA is represented in the figures as a black-colored strand.

FIG. 2 is a schematic illustration of certain embodiments of methods of the present disclosure in which the adapter-specific probe comprises a peptide nucleic acid (PNA) oligomer probe. A peptide nucleic acid oligomer (PNA) is comprised of a series of nucleotides that correspond to the target sequence or adapter sequence of interest (white—the Recognition Sequence), where the N-terminus of the PNA oligomer is labeled with a fluorescent dye (star) and the C-terminus of the PNA oligomer is labeled with a fluorescence quencher (rectangle). In aqueous solutions, the PNA oligomer folds in on itself and the close proximity of the quencher to the fluorescent dye results in negligible fluorescent signal. However, in the presence of a complementary DNA sequence (the adapter sequence which is complementary to the Recognition Sequence of the probe, represented as a white-colored strand), the PNA oligomer unwinds and binds to the target adapter sequence DNA (Activated Probe). When annealed, the unfolded-duplexed PNA oligomer increases the distance between the dye-quencher pair such that the quencher is no longer able to quench the dye's native fluorescence. This resulting fluorescence signal is quantified and represents a one-to-one relationship with the adapter modified DNA (Activated Probe). If no target sequence is recognized by the probe, the probe remains quenched and non-fluorescent (Inactive Probe).

FIG. 3 is a schematic illustration of certain embodiments of the methods of the present disclosure in which the adapter-specific probe comprises a molecular beacon probe. Molecular beacon probes are DNA oligomers organized into a hairpin loop comprised of 30-50 nucleotides that contain a loop region corresponding to the sequence of interest, as well as a stem region that is complementary to itself. The 5′-terminus of the molecular beacon stem is labeled with a fluorescent dye (star) and the 3′-stem terminus is labeled with a fluorescence quencher (rectangle). Natively, the stem sequence of 5-7 nucleotides aligns and anneals resulting in a close proximity of the quencher to the fluorescent dye resulting in negligible fluorescent signal. However, in the presence of a complementary DNA sequence (the adapter sequence which is complementary to the molecular beacon), the molecular beacon stem unanneals and the loop region is free to bind to the target DNA (the adapter sequence represented as a white-colored strand), which results in elongation of the molecular beacon probe DNA and alters the distance of the dye-quencher pair such that the quencher is no longer able to quench the dye's native fluorescence (Activated Probe). This resulting fluorescence signal is quantified and represents a one-to-one relationship with the adapter modified DNA. If no target sequence is recognized by the probe, the probe remains quenched and non-fluorescent (Inactive Probe).

FIG. 4 is a schematic illustration of certain embodiments of the methods provided herein in which the adapter-specific probe comprises a DNA flare probe. DNA flare probes are single stranded DNA (ssDNA) oligomers (gray) with incorporated fluorescent residues (star) protruding from one or more nucleotide. The fluorescence of the dyes is self-quenching in the single-strand native state (quenched probe), but turns-on when the probe anneals to its target sequence (white). This resulting fluorescence signal is quantified and represents a one-to-one relationship with the adapter-modified DNA (Activated Probe). If no target sequence is recognized by the probe, the probe remains quenched and non-fluorescent (Inactive Probe).

FIG. 5 is a schematic illustration of certain embodiments of the methods of the present disclosure in which the adapter-specific probe comprises a locked nucleic acid (LNA) probe pair. Locked nucleic acid (LNA) oligomers are used because their affinity towards the complementary ssDNA is greater than that of a standard dsDNA duplex. However, because LNA probes cannot be bent into a hairpin to form a beacon, two LNA probes are used. In this embodiment, a first LNA probe, referred to herein as LNA Probe #1, contains a fluorescent label (star) and a second LNA probe, referred to herein as LNA Probe #2, contains a fluorescence quencher (rectangle). Both LNA Probe #1 and LNA Probe #2 are added to the DNA sample containing the target sequence (white) and/or the non-complementary sequence (black). When LNA Probe #1 binds to the target sequence it maintains its fluorescence (Activated LNA Probe). Excess of LNA Probe #1 will be bound by LNA Probe #2, wherein the quencher moiety on LNA Probe #2 quenches the fluorescent signal from LNA Probe #1 (Inactive LNA Probe). The end result is that the signal will only be observable from the LNA Probe #1/target nucleic acid duplex (Activated LNA Probe). This signal is quantified and represents a one-to-one relationship with the adapter-modified DNA. If no target sequence is recognized by the probe, the probe will remain quenched and non-fluorescent (Inactive LNA Probe).

FIGS. 6A and 6B are graphic representations of the effect of the spacing of a fluorescent dye (fluorescein (FAM) in this example) on the fluorogenicity of a DNA flare probe. Increasing the spacing of the fluorophore (e.g., greater than 10 bases) had a detrimental effect on the signal-to-noise.

FIGS. 7A and 7B are graphic representations of the effect of the oligonucleotide size on the fluorescence of the DNA flares. Increasing the size of the oligonucleotide length of the DNA flare increased fluorescence without increasing background signal.

FIGS. 8A and 8B are graphic representations of the fluorogenicity of the DNA flares. All of the DNA flares shown are fluorogenic. At a spacing of less than 3 bases, fluorescent signal was lost even when bound to a complementary nucleic acid sequence. Although the total fluorescence intensity was reduced, the background was also diminished. The reduction in background led to a spacing of 4 bases between the fluorophore molecules (FAM) having a 40-fold increase in fluorescence (FIG. 8B).

### DETAILED DESCRIPTION OF THE INVENTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims.

To more clearly and concisely describe and point out the subject matter of the present disclosure, the following definitions are provided for specific terms, which are used in the following description and the appended claims. Throughout the specification, exemplification of specific terms should be considered as non-limiting examples.

As used in this specification, the words “a” or “an” mean at least one, unless specifically stated otherwise. In this specification, the use of the singular includes the plural unless specifically stated otherwise. For example, but not as a limitation, “a target nucleic acid” means that more than one target nucleic acid can be present; for example, one or more copies of a particular target nucleic acid species, as well as two or more different species of target nucleic acid. The term “and/or” means that the terms before and after the slash can be taken together or separately. For illustration purposes, but not as a limitation, “X and/or Y” can mean “X” or “Y” or “X” and “Y”.

It will be appreciated that there is an implied “about” prior to the temperatures, concentrations, times, etc. discussed in the present disclosure, such that slight and insubstantial deviations are within the scope of the present teachings herein. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or combinations thereof” as used herein refers to all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

The section headings used herein are for organizational purposes only and are not to be construed as limiting the desired subject matter in any way. All literature cited in the specification, including but not limited to, patents, patent applications, articles, books and treatises are expressly incorporated by reference in their entirety for any purpose. In the event that any of the incorporated literature contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### Definitions

The terms “annealing” and “hybridizing”, including, without limitation, variations of the root words “hybridize” and “anneal”, are used interchangeably and mean the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions may also contribute to duplex stability. Conditions under which primers and probes anneal to complementary sequences are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968).

In general, whether such annealing takes place is influenced by, among other things, the length of the complementary portions of the primers and/or probes and their corresponding binding sites in the target nucleic acid, or the corresponding complementary portions of a probe and its binding site; the pH; the temperature; the presence of mono- and divalent cations; the proportion of G and C nucleotides in the hybridizing region; the viscosity of the medium; and the presence of denaturants. Such variables influence the time required for hybridization. Thus, the preferred annealing conditions will depend upon the particular application. Such conditions, however, can be routinely determined by persons of ordinary skill in the art, without undue experimentation. Preferably, annealing conditions are selected to allow the primers and/or probes to selectively hybridize with a complementary sequence in the corresponding target nucleic acid, but not hybridize to any significant degree to different non-target nucleic acids or non-target nucleic acid sequences in the reaction mixture.

The term “selectively hybridize” and variations thereof, means that, under appropriate stringency conditions, a given sequence (for example, but not limited to, a primer or probe) anneals with a second sequence comprising a complementary string of nucleotides (for example, but not limited to, a target nucleic acid comprising a sequencing by synthesis adapter sequence), but does not anneal to undesired sequences, such as non-target nucleic acids, probes, or other primers. Typically, as the reaction temperature increases toward the melting temperature of a particular double-stranded sequence, the relative amount of selective hybridization generally increases and mis-priming generally decreases. In this specification, a statement that one sequence hybridizes or selectively hybridizes with another sequence encompasses situations where the entirety of both of the sequences hybridize or selectively hybridize to one another, and situations where only a portion of one or both of the sequences hybridizes or selectively hybridizes to the entire other sequence or to a portion of the other sequence.

The terms “denaturing” and “denaturation” as used herein refer to any process in which a double-stranded polynucleotide, including without limitation, DNA, peptide nucleic acids (PNA), locked nucleic acids (LNA), a genomic DNA (gDNA) fragment comprising at least one target nucleic acid, a double-stranded amplicon, or a polynucleotide comprising at least one double-stranded segment is converted to two single-stranded polynucleotides or to a single-stranded or substantially single-stranded polynucleotide, as appropriate. Denaturing a double-stranded polynucleotide includes, without limitation, a variety of thermal and chemical techniques which render a double-stranded nucleic acid single-stranded or substantially single-stranded, for example but not limited to, releasing the two individual single-stranded components of a double-stranded polynucleotide or a duplex comprising two oligonucleotides. Those in the art will appreciate that the denaturing technique employed is generally not limiting unless it substantially interferes with a subsequent annealing or enzymatic step of an amplification reaction, or in certain methods, the detection of a fluorescent signal.

The terms “complementarity” and “complementary” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementarity refers to the situation in which each nucleotide unit of one polynucleotide strand or region can hydrogen bond with each nucleotide unit of a second polynucleotide strand or region. “Less than perfect complementarity” refers to the situation in which some, but not all, nucleotide units of two strands or two units can hydrogen bond with each other.

As used herein, the term “nucleic acid-based probe” refers to synthetic or biologically produced nucleic acids (DNA, RNA, PNA, LNA, and the like) which, by design or selection, contain specific nucleotide sequences that allow them to hybridize, under defined stringencies, specifically (i.e., preferentially) to target nucleic acid sequences, and refers to adapter-specific probes and primer-dimer detection probes of the present disclosure. In the present teachings, the nucleic acid-based probes can be labeled, e.g., with a fluorescent dye, or a pair of labels comprising a fluorescent dye and a fluorescence quencher to enable detection. In some embodiments, the nucleic acid-based probe is at least partially quenched when not hybridized to a complementary sequence, and is at least partially unquenched when hybridized to a complementary sequence.

As used herein, the terms “quantitative PCR”, “qPCR”, “real-time PCR”, “reverse transcriptase PCR” and “rtPCR” refer to the use of the polymerase chain reaction (PCR) to quantify gene expression.

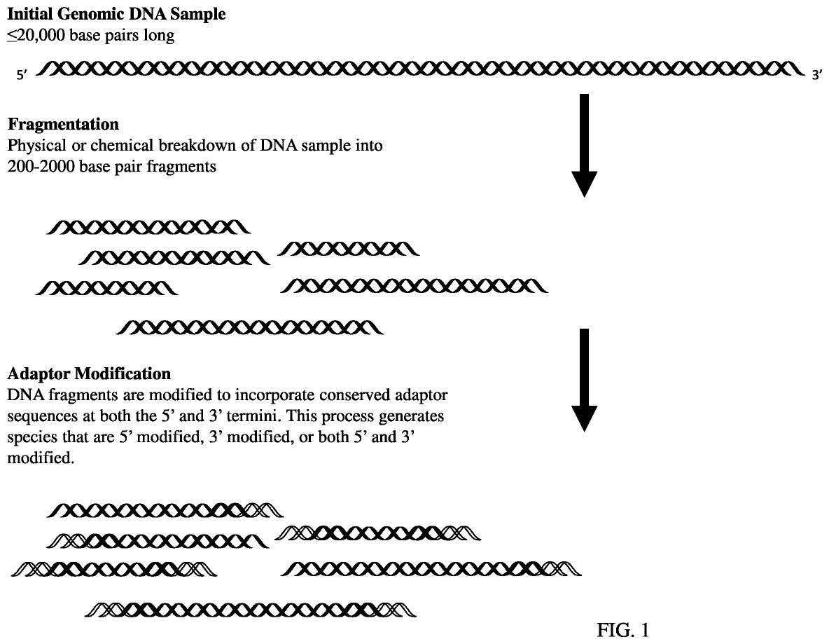

As used herein, the terms “adapter sequence” and “adapter” are interchangeable and refer to the nucleic acid sequence of oligonucleotides bound to the 5′-end and 3′-end of DNA fragments in a sequencing library as prepared by sequencing by synthesis methods, such as the methods used by the Illumina sequencing platform (Illumina, Inc., San Diego, Calif.) and described in U.S. Pat. No. 5,798,210 and Canard and Sarfati, Gene 148:1-6 (1994). The adapters are complementary to a lawn of oligonucleotides present on the surface of an Illumina sequencing flow cell, which is a glass slide with one or more physically separated lanes, each lane of which is coated with a lawn of surface-bound adapter-complementary oligonucleotides. Examples of such adapters include, but are not limited to, the Illumina TRUSEQ® Universal Adapter or a TRUSEQ® Indexed Adapter (Illumina, San Diego, Calif.). FIG. 1 depicts a schematic illustration of adapter modification of DNA using the Illumina sequencing by synthesis platform followed by quantification of the nucleic acid by either PCR or QUBIT™ quantitative assays (Thermo Fisher Scientific, Waltham, Mass.).

As used herein, the terms “target molecule”, “target nucleic acid molecule” and “target nucleic acid” are interchangeable and refer to a double-stranded or single-stranded nucleic acid molecule which is to be analyzed in the methods of the present disclosure. In the methods of the present disclosure, the target nucleic acid comprises at least one adapter sequence.

The target nucleic acid may be obtained from any source, and may comprise any number of different compositional components. For example, the target may be a nucleic acid (e.g., DNA or RNA) and may comprise nucleic acid analogs or other nucleic acid mimics. The target may be methylated, non-methylated, or both. The target may be bisulfite-treated and non-methylated cytosines converted to uracil. Further, it will be appreciated that “target nucleic acid” may refer to the target nucleic acid itself, as well as surrogates thereof, for example, amplification products and native sequences. The target molecules of the present teachings may be derived from any number of sources, including without limitation, viruses, archaea, protists, prokaryotes and eukaryotes, for example, but not limited to, plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells and lysed cells. It will be appreciated that target nucleic acids may be isolated from samples using any of a variety of procedures known in the art. It will be appreciated that target nucleic acids may be cut or sheared prior to analysis, including the use of such procedures as mechanical force, sonication, restriction endonuclease cleavage, or any method known in the art. In general, the target nucleic acids of the present teachings will be double-stranded, though in some embodiments the target nucleic acids may be single-stranded.

As used herein, the terms “hairpin” and “stem-loop” are interchangeable and are used to indicate the structure of an oligonucleotide in which one or more portions of the oligonucleotide form base pairs with one or more other portions of the oligonucleotide. When the two portions are base paired to form a double-stranded portion of the oligonucleotide, the double-stranded portion may be referred to as a stem. Thus, depending on the number of complementary portions used, a number of stems may be formed; preferably one stem is formed for molecular beacon probes.

As used herein, the terms “polynucleotide”, “oligonucleotide,” and “nucleic acid” are used interchangeably and refer to single-stranded and double-stranded polymers of nucleotide monomers, including without limitation, 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺, and the like. A polynucleotide may be composed entirely of deoxyribonucleotides, entirely of ribonucleotides, or chimeric mixtures thereof and may include nucleotide analogs. Oligonucleotides, as used herein, include natural nucleic acid molecules (i.e., DNA and RNA) as well as non-natural or derivative molecules such as peptide nucleic acids (PNA), locked nucleic acids (LNA), phosphothioate containing nucleic acids, phosphonate containing nucleic acids and the like. The nucleotide monomer units may comprise any of the nucleotides described herein, including, but not limited to, nucleotides and/or nucleotide analogs. Polynucleotides typically range in size from a few monomeric units, e.g., 5-40 when they are sometimes referred to in the art as oligonucleotides or oligos, to several thousand or a million of monomeric nucleotide units. Unless denoted otherwise, whenever a polynucleotide sequence is represented, it will be understood that the nucleotides are in the 5′-to-3′ order from left to right and that “A” denotes deoxyadenosine, “C” denotes deoxycytosine, “G” denotes deoxyguanosine, “T” denotes deoxythymidine, and “U” denotes deoxyuridine, unless otherwise noted. As used herein, oligonucleotides can comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The term “nucleotide” refers to a phosphate ester of a nucleoside, e.g., triphosphate esters, wherein the most common site of esterification is the hydroxyl group attached at the C-5 position of the pentose.

The term “nucleoside” refers to a compound consisting of a purine, deazapurine, or pyrimidine nucleoside base, e.g., adenine, guanine, cytosine, uracil, thymine, deazaadenine, deazaguanosine, and the like, linked to a pentose at the 1′-position, including 2′-deoxy and 2′-hydroxyl forms. When the nucleoside base is purine or 7-deazapurine, the pentose is attached to the nucleobase at the 9-position of the purine or deazapurine; when the nucleobase is pyrimidine, the pentose is attached to the nucleobase at the 1-position of the pyrimidine.

For use as described herein, one or more detectable labels and/or quenching agents may be attached to one or more probes (e.g., detectable label). The detectable label may emit a signal when free or when bound to one of the target nucleic acids. The detectable label may also emit a signal when in proximity to another detectable label. Detectable labels may also be used with energy transfer molecules such that the signal emitted from one label is absorbed and re-emitted from a second label. Detectable labels may also be used with quencher molecules such that the signal is only detectable when not in sufficiently close proximity to the quencher molecule. For instance, in some embodiments, the assay system may cause the detectable label to be liberated from the quenching molecule. Any of several detectable labels may be used to label the probes used in the methods described herein. When using more than one detectable label, each should differ in their spectral properties such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and Förester Resonance Energy Transfer molecules (FRET) where the fluorescent signal from one label is absorbed and re-emitted by a second label, and the like. Suitable detectable labels may include fluorophores, for example, a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in U.S. Patent Application Publication Nos. 2002/0077487 and 2002/0064794), a carbocyanine (including any corresponding compounds in U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 6,664,047; 6,974,873; 6,977,305; PCT Publication Nos. WO 02/26891, WO 97/40104, WO 99/51702, WO 01/21624; and European Patent Application Publication No. EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 6,716,979 and 5,451,343), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs. Other detectable labels may also be used as would be known to those of skill in the art.

For use as described herein, the terms “quencher”, “quencher molecule” and “fluorescence quencher” are interchangeable and refer to any molecule that absorbs the natural fluorescence of a molecule rendering the fluorescence non-observable. Moreover, a quencher molecule can be replaced with a FRET accepter molecule. For purposes of the assay, these concepts are interchangeable.

Methods of Quantifying Nucleic Acids and Probes for Use Therein:

Next Generation Sequencing (NGS) is a rapidly growing field of study. The newfound speed with which genomic information can be accessed has revolutionized the study of biology. Indeed, sequencing information that once took years to obtain can now be done within a few days. Several primary sequencing technologies exist; however, the platform provided by Illumina, Inc. (San Diego, Calif.) is currently the market leader for laboratory researchers. In the sample preparation of the Illumina workflow, users need to quantify their DNA sample at several points: prior to fragmentation, before adapter ligation, and after ligation before loading the samples onto the sequencing instrument. The quantification step is critical, because while the technology does allow the rapid sequencing of a genome, the run itself is costly and the data processing time is lengthy. The most trusted methods for performing quantitation of the DNA samples are fluorescence using the QUBIT™ Fluorometer and QUBIT™ quantitation assays (Thermo Fisher Scientific, Waltham, Mass.) and quantitative PCR (qPCR). Each method has its own merits and drawbacks.

The QUBIT™ quantitation assays provide results in less than two minutes and can do so in the presence of competing RNA and single stranded (ssDNA) impurities. The technology relies on an intercalating fluorogenic dye that emits a fluorescent signal when it is bound to DNA, and only DNA. The QUBIT™ quantitation assay quantifies the total DNA in the sample in terms of mass per volume (ng/μL). Unfortunately, NGS users need to convert these units into molarity (molecules per volume) for proper instrument loading. This conversion is typically done through the use of a gel electrophoresis instrument that determines the average size of the DNA in the sample. A typical error for this measurement is ±50%. Therefore, due to error in the measured DNA size, the molar value the users are obtaining for their often precious DNA sample is at best 50% accurate. So while the QUBIT™ quantitative assay itself is very robust and provides reliable data, unfortunately the NGS application requires a data manipulation that inserts 5-times more error than the typical user is comfortable with.

The alternative to QUBIT™-based quantitation is real-time PCR (rtPCR), also known as quantitative PCR (qPCR). This is a technique that amplifies specific DNA molecules based on the presence of a particular sequence. Only DNA containing the primer sequence will be amplified. The resulting DNA is quantified using a fluorescence report-out that reads based on the primer sequence, so only DNA containing the proper sequence can be quantified. This is very valuable because the Illumina sequencing instrument will only sequence DNA containing the same sequence motif, allowing users to be more exact in the sample preparation and have greater confidence in their results. The tradeoff for this approach is the 6 hour time commitment for the setup and experiment.

All samples sequenced with an Illumina instrument are modified with adapter DNA, herein referred to as a sequencing by synthesis adapter or adapter sequence, during sample preparation prior to sample sequencing. These special DNA adapter sequences are incorporated into the users' DNA sample allowing the sample to interface with the Illumina sequencing technology (See, FIG. 1 for a schematic illustration; also described in U.S. Pat. No. 5,798,210 and Canard and Sarfati, Gene 148:1-6 (1994)). Sample DNA that does not contain the adapter sequence will not interface with the instrument and therefore cannot be sequenced. The assay methods for detecting and/or quantitating nucleic acids provided herein utilize an adapter-specific probe which is a nucleic acid-based probe that is complementary all or a portion of to the conserved Illumina adapter sequence allowing for target specificity. Upon binding, the adapter-specific probe emits a fluorescence signal that can be measured. Advantageously, the measured fluorescence signal is directly proportional to the number of adapter-modified DNA molecules. Only DNA containing the adapter sequence will trigger the probe's fluorescence response. This allows users to quantify only the DNA in their sample that has been modified with the adapter sequence and is capable of being sequenced. Additionally, because the fluorescence signal is in a one-to-one ratio with the adapter modified DNA; the read-out units will be in molarity. This is ideal for the user as these units are required to continue with the sequencing application. Other units would have to be converted and can introduce error into the measurement.

An additional advantageous feature of the methods provided herein is the optional use of an additional nucleic acid-based probe referred to herein as a “primer-dimer detection probe” which is detectably distinct from (e.g., orthogonal to) the adapter-specific probe. The primer-dimer detection probe allows detection of “primer-dimer” artifacts in the methods provided herein. Without wishing to be bound by theory, these “primer-dimer” artifacts arise from a poor modification step when the adapter sequences are added to the target DNA. The presence of too much of the primer-dimer DNA can interfere with the sequencing and lead to poor or failed experiments. Typically the primer-dimer detection probe is comprised of the same technology as the adapter-specific probe (e.g., PNA probe, molecular beacon, DNA flare or LNA probe pair), but the fluorescence of the primer-dimer detection probe is detectably distinct from the fluorescence of the adapter-specific probe (e.g., the fluorescence of the probes is orthogonal). Ideally, the adapter-specific probe and the primer-dimer detection probe can be added to the same sample and the user will receive two read outs: one from the adapter-specific probe and the second from the primer-dimer detection probe. Alternatively, the user can use two separate sample tubes and detect each probe separately. For example, signal from the first sample tube using the adapter-specific probe will measure the target DNA used for sequencing, and signal from the second sample tube using the primer-dimer detection probe will measure the primer-dimers.

In general, the methods of the present disclosure utilize unique nucleic acid-based probes based on oligonucleotides comprising a peptide nucleic acid (PNA) oligomer, a molecular beacon, a DNA flare or locked nucleic acid (LNA) probe pair. Each probe technology relies on a fluorescence signal that turns on when the nucleic acid-based probe is annealed to the target of interest. Advantageously, this signal is based on a one-to-one ratio of the probe to the target, and when no target sequence is available, the probe's fluorescence remains quenched. In certain embodiments, the target DNA sequence is the conserved Illumina adapter sequence. Examples of such adapters include, but are not limited to, the Illumina TRUSEQ® Universal Adapter or a TRUSEQ® Indexed Adapter (Illumina, San Diego, Calif.). In certain embodiments, an additional probe, the “primer-dimer detection probe”, is used to target the DNA “primer-dimer” sequence which occurs where the adapter sequences form dimers rather than incorporating into the users' DNA sample.

In certain embodiments of the present disclosure, the assay can simultaneously detect the adapter sequence modification at the 3′-end and the 5′-end of the target DNA. An adapter-specific probe that is complementary to the 3′-end of the sequencing by synthesis adapter specifically targets the 3′-end of the adapter sequence modification of the target nucleic acid. An adapter-specific probe that is complementary to the 5′-end of the sequencing by synthesis adapter specifically targets the 5′-end of the adapter sequence modification of the target nucleic acid. The fluorescent reporting moieties of these probes will be orthogonal to each other and the primer-dimer probe allowing the probes to be multiplexed or assayed separately.

In certain embodiments of the present disclosure, a method for detecting a nucleic acid in a sample is provided, the method comprising:
a) combining a nucleic acid probe with the sample to prepare a probe-nucleic acid mixture;
b) incubating the probe-nucleic acid mixture for a sufficient amount of time for the nucleic acid probe to hybridize to the nucleic acid in the sample to form a probe-nucleic acid complex;
c) illuminating the probe-nucleic acid complex with an appropriate wavelength of light to generate a detectable optical response; and
d) detecting the detectable optical response thereby detecting the nucleic acid.

In certain embodiments of the present disclosure, a method for quantifying a nucleic acid in a sample is provided, the method comprising:
a) combining a nucleic acid probe with the sample to prepare a probe-nucleic acid mixture;
b) incubating the probe-nucleic acid mixture for a sufficient amount of time for the nucleic acid probe to hybridize to the nucleic acid in the sample to form a probe-nucleic acid complex;
c) illuminating the probe-nucleic acid complex with an appropriate wavelength of light to generate a detectable optical response;
d) detecting the detectable optical response; and
e) comparing the detectable optical response with a known quantity of nucleic acid thereby quantifying the nucleic acid in the sample.

In certain embodiments of the present disclosure, a method of detecting a target nucleic acid in a sample is provided, the method comprising:
a) providing a sample containing a target nucleic acid comprising at least one sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence; and
c) incubating the target-probe mixture for a sufficient amount of time to allow the adapter-specific probe to hybridize to the target nucleic acid to form a probe-target complex.

In certain embodiments, the method further comprises:
d) illuminating the probe-target complex with an appropriate wavelength of light to generate a detectable optical response; and
e) detecting the detectable optical response thereby detecting the target nucleic acid in the sample.

In certain preferred embodiments, the adapter-specific probe is complementary to the 3′-end or the 5′-end of the sequencing by synthesis adapter sequence. In certain preferred embodiments, the detectable optical response is proportional to the amount of target nucleic acid present in the sample.

In certain embodiments of the present disclosure, a method of detecting a target nucleic acid in a sample is provided, the method comprising:
a) providing a sample containing a target-probe mixture, wherein the target-probe mixture comprises i) a target nucleic acid comprising at least one sequencing by synthesis adapter sequence, and ii) an adapter-specific probe comprising a nucleic acid sequence that is complementary to the sequencing by synthesis adapter sequence;
b) illuminating the target-probe mixture with an appropriate wavelength of light to generate a detectable optical response; and
c) detecting the detectable optical response thereby detecting the target nucleic acid in the sample.

In certain additional embodiments of the present disclosure, a method of detecting a target nucleic acid in a sample is provided, the method comprising:
a) providing a sample containing one or more target-probe mixtures, wherein the one or more target-probe mixtures comprises i) a target nucleic acid comprising at least one sequencing by synthesis adapter sequence, and ii) one or more adapter-specific probe comprising a nucleic acid sequence that is complementary to the sequencing by synthesis adapter sequence;
b) illuminating the one or more target-probe mixture with an appropriate wavelength of light to generate a detectable optical response; and
c) detecting the detectable optical response thereby detecting the target nucleic acid in the sample.

In certain embodiments, the sample contains two target-probe mixtures. In certain embodiments, the sample contains three target-probe mixtures. In certain preferred embodiments, the adapter-specific probe is complementary to the 3′-end or the 5′-end of the sequencing by synthesis adapter sequence.

In certain embodiments of the present disclosure, a method of detecting a target nucleic acid in a sample is provided, the method comprising:
a) providing a sample containing a target nucleic acid comprising at least one sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence; and
d) incubating the first target-probe mixture and the second target-probe mixture for a sufficient amount of time to allow the first adapter-specific probe and the second adapter-specific probe to hybridize to the target nucleic acid to form a first probe-target complex and a second probe-target complex;
wherein the first adapter-specific probe and the second adapter-specific probe are detectably distinct.

In certain embodiments, the methods further comprise:
e) illuminating the first probe-target complex and the second probe-target complex with an appropriate wavelength of light to generate a first detectable optical response and a second detectable optical response; and
f) detecting the first detectable optical response and the second detectable optical response thereby detecting the target nucleic acid in the sample.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the adapter-specific probe is complementary to the 3′-end or the 5′-end of the sequencing by synthesis adapter sequence.

In certain embodiments of the present disclosure, a method of quantifying a target nucleic acid in a sample is provided, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence; and
c) incubating the target-probe mixture for a sufficient amount of time to allow the adapter-specific probe to hybridize to the target nucleic acid to form a probe-target complex.

In certain embodiments, the method further comprises:
d) illuminating the probe-target complex with an appropriate wavelength of light to generate a detectable optical response;
e) detecting the detectable optical response; and
f) comparing the detectable optical response with a known quantity of nucleic acid thereby quantifying the target nucleic acid in the sample.

In certain preferred embodiments, the adapter-specific probe is complementary to the 3′-end or the 5′-end of the sequencing by synthesis adapter sequence. In certain preferred embodiments, the detectable optical response is proportional to the amount of target nucleic acid present in the sample.

In certain embodiments of the present disclosure, a method of quantifying a target nucleic acid in a sample is provided, the method comprising:
a) providing a sample containing a target nucleic acid comprising at least one sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence; and
d) incubating the first target-probe mixture and the second target probe mixture for a sufficient amount of time to allow the first adapter-specific probe and the second adapter-specific probe to hybridize to the target nucleic acid to form a first probe-target complex and a second probe-target complex;

wherein the first adapter-specific probe and the second adapter-specific probe are detectably distinct.

In certain embodiments, the method further comprises:
e) illuminating the first probe-target complex and the second probe-target complex with an appropriate wavelength of light to generate a first detectable optical response and a second detectable optical response; and
f) detecting the first detectable optical response and the second detectable optical response thereby detecting the target nucleic acid in the sample;
g) comparing the first detectable optical response and the second detectable optical response with a known quantity of nucleic acid thereby quantifying the target nucleic acid in the sample.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the detectable optical response is proportional to the amount of target nucleic acid present in the sample.

In certain embodiments, a method of quantifying a target nucleic acid in a sample is provided, the method comprising:
a) providing a sample containing a target-probe mixture, wherein the target-probe mixture comprises i) a target nucleic acid comprising a sequencing by synthesis adapter sequence, and ii) an adapter-specific probe comprising a nucleic acid sequence that is complementary to the sequencing by synthesis adapter sequence;
b) illuminating the target-probe mixture with an appropriate wavelength of light to generate a detectable optical response;
c) detecting the detectable optical response; and
d) comparing the detectable optical response with a known quantity of nucleic acid thereby quantifying the target nucleic acid in the sample.

In certain embodiments, a method of quantifying a target nucleic acid in a sample is provided, the method comprising:
a) providing a sample containing one or more target-probe mixtures, wherein the one or more target-probe mixtures comprises i) a target nucleic acid comprising at least one sequencing by synthesis adapter sequence, and ii) one or more adapter-specific probes comprising a nucleic acid sequence that is complementary to the sequencing by synthesis adapter sequence;
b) illuminating the one or more target-probe mixture with an appropriate wavelength of light to generate a detectable optical response; and
c) detecting the detectable optical response thereby detecting the target nucleic acid in the sample.

In certain embodiments, the sample contains two target-probe mixtures. In certain embodiments, the sample contains three target-probe mixtures. In certain preferred embodiments, the adapter-specific probe is complementary to the 3′-end or the 5′-end of the sequencing by synthesis adapter sequence.

In certain embodiments of the present disclosure, a method of detecting a target nucleic acid in a sample is provided, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence;
c) heating the target-probe mixture for a sufficient amount of time to allow the target nucleic acid to denature;
d) cooling the target-probe mixture for a sufficient amount of time to allow the adapter-specific probe to hybridize to the target nucleic acid to create a probe-target complex;
e) illuminating the probe-target complex with an appropriate wavelength of light to generate a detectable optical response; and
f) detecting the detectable optical response thereby detecting the target nucleic acid in the sample.

In certain preferred embodiments, the adapter-specific probe is complementary to the 3′-end or the 5′-end of the sequencing by synthesis adapter. In certain preferred embodiments, the detectable optical response is proportional to the amount of target nucleic acid present in the sample.

In certain embodiments of the present disclosure, a method of detecting a target nucleic acid in a sample is provided, the method comprising:
a) providing a sample containing a target nucleic acid comprising a sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence;
d) heating the first target-probe mixture and the second target-probe mixture for a sufficient amount of time to allow the target nucleic acid to denature;
e) cooling the first target-probe mixture and the second target-probe mixture for a sufficient amount of time to allow the first adapter-specific probe and the second adapter-specific probe to hybridize to the target nucleic acid to create a first probe-target complex and a second probe-target complex;
f) illuminating the first probe-target complex and the second probe-target complex with an appropriate wavelength of light to generate a first detectable optical response and a second detectable optical response; and
g) detecting the first detectable optical response and the second detectable optical response thereby detecting the target nucleic acid in the sample, wherein the first adapter-specific probe and the second adapter-specific probe are detectably distinct.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5 ‘-end of the sequencing by synthesis adapter sequence. In certain embodiments, the first adapter-specific probe is complementary to the 5’-end of the sequencing by synthesis adapter sequence and the second adapter-specific probe is complementary to the 3′-end of the sequencing by synthesis adapter sequence. In certain embodiments, step (b) and step (c) are performed simultaneously.

In certain embodiments, step (b) and step (c) are performed sequentially. In certain preferred embodiments, the detectable optical response is proportional to the amount of target nucleic acid present in the sample.

In certain embodiments, a method of quantifying a target nucleic acid in a sample is provided, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence;
c) heating the target-probe mixture for a sufficient amount of time to allow the target nucleic acid to denature;
d) cooling the target-probe mixture for a sufficient amount of time to allow the adapter-specific probe to hybridize to the target nucleic acid to create a probe-target complex;
e) illuminating the probe-target complex with an appropriate wavelength of light to generate a detectable optical response;
f) detecting the detectable optical response; and
g) comparing the detectable optical response with a known quantity of nucleic acid thereby quantifying the target nucleic acid in the sample.

In certain preferred embodiments, the adapter-specific probe is complementary to the 3′-end or the 5′-end of the sequencing by synthesis adapter. In certain preferred embodiments, the detectable optical response is proportional to the amount of target nucleic acid present in the sample.

In certain embodiments of the present disclosure, a method of quantifying a target nucleic acid in a sample is provided, the method comprising:
a) providing a sample containing a target nucleic acid comprising a sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence;
d) heating the first target-probe mixture and the second target-probe mixture for a sufficient amount of time to allow the target nucleic acid to denature;
e) cooling the first target-probe mixture and the second target-probe mixture for a sufficient amount of time to allow the first adapter-specific probe and the second adapter-specific probe to hybridize to the target nucleic acid to create a first probe-target complex and a second target-probe complex;
f) illuminating the first probe-target complex and the second target-probe complex with an appropriate wavelength of light to generate a first detectable optical response and a second detectable optical response;
g) detecting the first detectable optical response and the second detectable optical response; and
h) comparing the first detectable optical response and the second detectable optical response with a known quantity of nucleic acid thereby quantifying the target nucleic acid in the sample, wherein the first adapter-specific probe and the second adapter-specific probe are detectably distinct.

In certain preferred embodiments, the first region of the sequencing by synthesis adapter sequence is a 3′-end region and the second region of the sequencing by synthesis adapter sequence is a 5′-end region. In certain preferred embodiments, the first region of the sequencing by synthesis adapter sequence is a 5′-end region and the second region of the sequencing by synthesis adapter sequence is a 3′-end region. In certain preferred embodiments, the first adapter-specific probe is complementary to the 3′-end of the sequencing by synthesis adapter sequence and the second adapter-specific probe is complementary to the 5′-end of the sequencing by synthesis adapter sequence. In certain embodiments, the first adapter-specific probe is complementary to the 5′-end of the sequencing by synthesis adapter sequence and the second adapter-specific probe is complementary to the 3′-end of the sequencing by synthesis adapter sequence. In certain embodiments, step (b) and step (c) are performed simultaneously. In certain embodiments, step (b) and step (c) are performed sequentially. In certain preferred embodiments, the detectable optical response is proportional to the amount of target nucleic acid present in the sample.

In certain embodiments of the methods provided herein, the target nucleic acid is double-stranded DNA.

In certain embodiments of the methods provided herein, the adapter-specific probe comprises an oligonucleotide sequence that is complementary to the sequencing by synthesis adapter sequence, or a portion thereof, and a fluorescent dye. In certain embodiments of the methods provided herein, the adapter-specific probe further comprises a fluorescence quencher. In certain embodiments of the methods provided herein, the adapter-specific probe is a peptide nucleic acid (PNA) oligomer probe, a molecular beacon probe, a DNA flare probe or a locked nucleic acid (LNA) probe pair. In certain embodiments, the fluorescent dye and/or quencher are directly attached to one or more nucleotides of the adapter-specific probe. In certain embodiments, the fluorescent dye and/or quencher are covalently attached to one or more nucleotides of the adapter-specific probe.

In certain embodiments of the methods provided herein, the adapter-specific probe is a peptide nucleic acid (PNA) oligomer comprising a fluorescent attached to the N-terminus of the PNA oligomer, a quencher attached to the C-terminus of the PNA oligomer, and a series of nucleotides that are complementary to a nucleic acid sequence of the sequencing by synthesis adapter. In certain embodiments, the PNA oligomer comprises 8-12 nucleotides that are complementary to a nucleic acid sequence of the sequencing by synthesis adapter.

In certain embodiments of the methods provided herein, the adapter-specific probe is a molecular beacon comprising a fluorescent dye attached to the 5′-end of the molecular beacon, a fluorescence quencher attached to the 3′-end of the molecular beacon, and 18 nucleotides in the loop portion that are complementary to a nucleic acid sequence of the sequencing by synthesis adapter.

In certain embodiments of the methods provided herein, the adapter-specific probe is a single-stranded DNA flare comprising a fluorescent dye covalently attached to one or more nucleotides, and 28 nucleotides that are complementary to a nucleic acid sequence of the sequencing by synthesis adapter. In certain embodiments, the DNA flare probe is double stranded.

In certain embodiments of the methods provided herein, the adapter-specific probe is a locked nucleic acid (LNA) probe pair comprising a first LNA probe comprising a fluorescent dye attached to either the 3′- or 5′-end of the first probe and a series of nucleotides that are complementary to a nucleic acid sequence of the sequencing by synthesis adapter, and a second LNA probe comprising a fluorescence quencher attached to either the 3′- or 5′-end of the second probe and a series of nucleotides that are complementary to the nucleic acid sequence of the first LNA probe (e.g., complementary to the first LNA probe).

In certain embodiments of the methods provided herein, the methods further comprise adding a primer-dimer detection probe to the target-probe mixture, wherein the primer-dimer detection probe has a detectable optical response that is distinguishable from the detectable optical response of the adapter-specific probe.

In certain embodiments of the methods provided herein, the detecting step is performed by fluorimetry. In certain embodiments of the methods provided herein, the sample is in a microfuge tube or a multi-well plate. In certain preferred embodiments, the detecting step is performed on a QUBIT™ fluorometer (Thermo Fisher Scientific, Waltham, Mass.).

In certain embodiments of the methods provided herein, the sequencing by synthesis adapter is a TRUSEQ® Universal Adapter or a TRUSEQ® Indexed Adapter (Illumina, San Diego, Calif.).

Peptide Nucleic Acid (PNA) Oligomer Nucleic Acid-Based Probes:

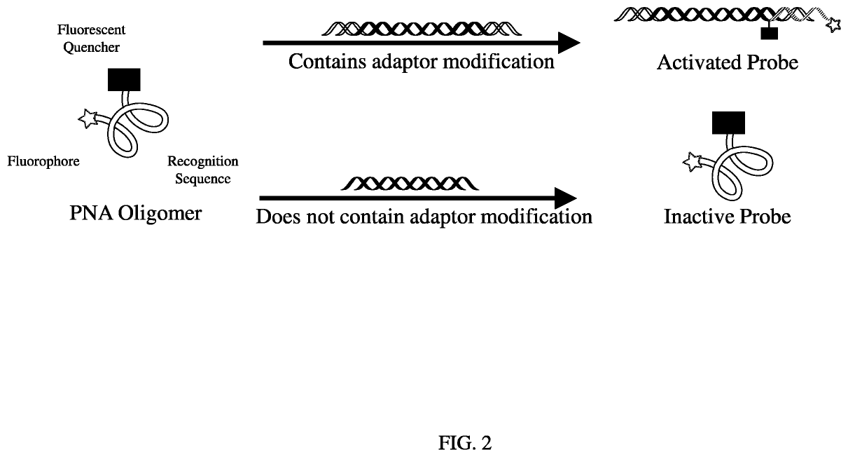

As shown in FIG. 2, in certain embodiments of the methods described herein, the adapter-specific probe comprises a peptide nucleic acid (PNA) oligomer nucleic acid-based probe that corresponds to the target nucleic acid sequence of interest, also known as the sequencing by synthesis adapter sequence, where the N-terminus of the PNA oligomer is labeled with a fluorescent dye and the C-terminus of the PNA oligomer is labeled with a quencher. In aqueous solutions, the PNA oligomer folds in on itself and the close proximity of the quencher to the fluorescent dye results in negligible fluorescent signal. However, in the presence of a complementary DNA sequence, for example the Illumina adapter sequence, the PNA oligomer unwinds and binds to the target DNA. When annealed, the unfolded-duplexed PNA oligomer increases the distance between the dye-quencher pair such that the quencher is no longer able to quench the dye's native fluorescence. This resulting fluorescence signal is quantified using the methods disclosed herein and advantageously represents a one-to-one relationship with the adapter modified DNA. If no target sequence is recognized by the probe, the probe remains quenched and non-fluorescent.

In certain embodiments, adapter-specific probes are provided, which probes comprise peptide nucleic acid (PNA) oligomers comprising a fluorescent dye attached to the N-terminus of the PNA oligomer, a quencher attached to the C-terminus of the PNA oligomer, and a series of nucleotides that are complementary to the nucleic acid sequence of the sequencing by synthesis adapter. In certain embodiments, the PNA oligomer comprises 8-12 nucleotides that are complementary to the nucleic acid sequence of the sequencing by synthesis adapter.

Molecular Beacon Nucleic Acid-Based Probes:

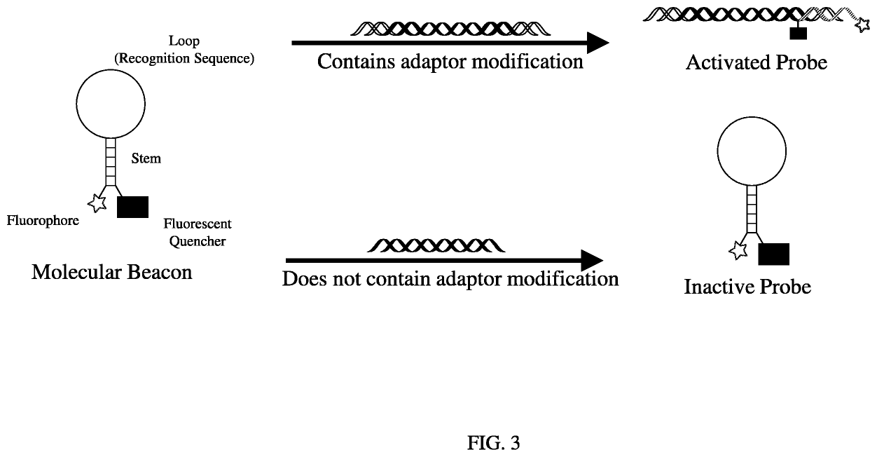

As shown in FIG. 3, in certain embodiments of the methods described herein, the adapter-specific probe comprises a molecular beacon nucleic acid-based probe, which is a DNA oligomer organized into a hairpin loop comprised of 30-50 nucleotides that contain a loop region corresponding to the sequence of interest, as well as a stem region that is complementary. The 5′-terminus of the stem is labeled with a fluorescent dye and the 3′-stem terminus is labeled with a quencher. Natively, the stem sequence of 5-7 nucleotides aligns resulting in a close proximity of the quencher to the fluorescent dye results in negligible fluorescent signal. However, in the presence of a complementary DNA sequence, for example the Illumina adapter sequence, the molecular beacon stem unanneals and the loop region is free to bind to the target DNA. This results in elongation of the DNA and alters the distance of the dye-quencher pair such that the quencher is no longer able to quench the dye's native fluorescence. This resulting fluorescence signal is quantified using the methods described herein and advantageously represents a one-to-one relationship with the adapter modified DNA. If no target sequence is recognized by the probe, it remains quenched and non-fluorescent.

In certain embodiments, adapter-specific probes are provided, which probes comprise molecular beacon probes comprising a fluorescent dye attached to the 5′-end of the molecular beacon, a quencher attached to the 3′-end of the molecular beacon, and 18 nucleotides in the loop portion that are complementary to the nucleic acid sequence of the sequencing by synthesis adapter.

DNA Flare Nucleic Acid-Based Probes:

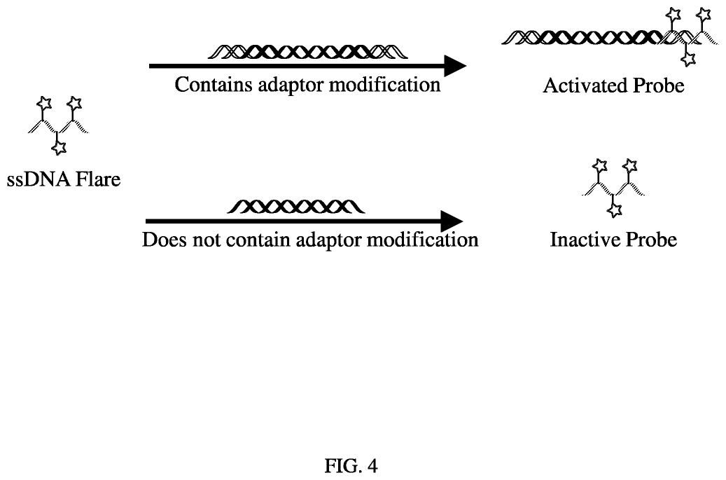

As shown in FIG. 4, in certain embodiments of the methods described herein, the adapter-specific probe comprises a DNA flare nucleic acid-based probe which comprises ssDNA oligomers comprising incorporated fluorescent residues attached to nucleotides. The fluorescence of the dyes is self-quenching in the single-strand native state, but turns-on when the probe anneals to its target sequence. This resulting fluorescence signal is quantified using the methods described herein and advantageously represents a one-to-one relationship with the adapter modified DNA. If no target sequence is recognized by the probe, the probe remains quenched and non-fluorescent.

In certain embodiments, adapter-specific probes are provided, which probes comprise single-stranded DNA flare probes comprising a fluorescent dye covalently attached to one or more nucleotides, and 28 nucleotides that are complementary to a nucleic acid sequence of the sequencing by synthesis adapter. In certain embodiments, the DNA flare probes provided herein have a separation of at least 4 bases between the fluorescent dye molecules. In certain embodiments, the DNA flare probes have a separation of at least 5 bases, at least 6 bases, at least 7 bases, at least 8 bases, at least 9 bases or at least 10 bases between the fluorescent dye molecules. In certain embodiments, the oligonucleotide sequence is complementary to all or a portion of the nucleic acid sequence of the sequencing by synthesis adapter.

Locked Nucleic Acid (LNA) Probe Pairs:

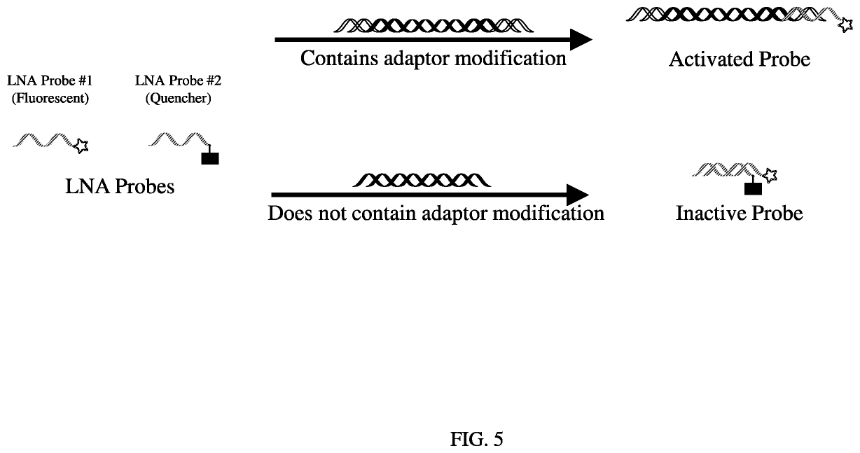

As shown in FIG. 5, certain embodiments described herein the adapter-specific probe comprises a locked nucleic acid (LNA) probe pair comprising a first LNA probe (referred to as LNA Probe #1) comprising a fluorescent dye attached to either the 3′- or 5′-end of the first probe and a series of nucleotides that are complementary to the nucleic acid sequence of the sequencing by synthesis adapter, and a second LNA probe (referred to as LNA Probe #2) comprising a fluorescence quencher attached to either the 3′- or 5′-end of the second probe and a series of nucleotides that are complementary to the nucleic acid sequence of the first LNA probe (e.g., complementary to LNA Probe #1). In certain embodiments, the series of nucleotides that is complementary to the nucleic acid sequence of the sequencing by synthesis adapter is 8-12 nucleotides. In this embodiment, LNA Probe #1 contains a fluorescent label and LNA Probe #2 contains a fluorescence quencher. Both LNA Probe #1 and LNA Probe #2 are added to the DNA sample containing the target sequence and/or the non-complementary sequence. When LNA Probe #1 binds to the target sequence it maintains its fluorescence. Excess of LNA Probe #1 will be bound by LNA Probe #2, wherein the quencher moiety on LNA Probe #2 quenches the fluorescent signal from LNA Probe #1. The end result is that the signal will only be observable from the LNA Probe #1/target nucleic acid duplex. This signal is quantified and represents a one-to-one relationship with the adapter-modified DNA. If no target sequence is recognized by the probe, the probe will remain quenched and non-fluorescent.

Primer-Dimer Detection Probes:

In certain embodiments, a primer-dimer detection probe is provided, whose target sequence is the overlapping region between the two attached primer sequences. In certain embodiments, the primer-dimer detection probe comprises an oligonucleotide sequence that is complementary to a nucleic acid sequence of a sequencing by synthesis primer-dimer and a fluorescent dye. In certain embodiments, the primer-dimer detection probe further comprises a quencher. In certain embodiments, the primer-dimer detection probe comprises a peptide nucleic acid (PNA) oligomer probe, a molecular beacon probe, a DNA flare probe or a locked nucleic acid (LNA) probe pair. In certain embodiments, the fluorescent dye and/or quencher are directly attached to one or more nucleotides of the primer-dimer detection probe. In certain embodiments, the fluorescent dye and/or quencher are covalently attached to one or more nucleotides of the primer-dimer detection probe.

In certain embodiments of the methods provided herein, the primer-dimer detection probe comprises a peptide nucleic acid (PNA) oligomer comprising a fluorescent dye attached to the N-terminus of the PNA oligomer, a quencher attached to the C-terminus of the PNA oligomer, and a series of nucleotides that are complementary to the nucleic acid sequence of the sequencing by synthesis primer-dimer. In certain embodiments, the PNA oligomer comprises 8-12 nucleotides that are complementary to the nucleic acid sequence of the sequencing by synthesis primer-dimer. The fluorescent dye and quencher used in the primer-dimer detection probe are detectably distinct from the fluorescent dye and quencher used in the adapter-specific probe.

In certain embodiments of the methods provided herein, the primer-dimer detection probe comprises a molecular beacon comprising a fluorescent dye attached to the 5′-end of the molecular beacon, a quencher attached to the 3′-end of the molecular beacon, and 18 nucleotides in the loop portion that are complementary to the nucleic acid sequence of the sequencing by synthesis primer-dimer. The fluorescent dye and quencher used in the primer-dimer detection probe are detectably distinct from the fluorescent dye and quencher used in the adapter-specific probe.

In certain embodiments of the methods provided herein, the primer-dimer detection probe comprises a DNA flare probe comprising a fluorescent dye covalently attached to one or more nucleotides, and 28 nucleotides that are complementary to the nucleic acid sequence of the sequencing by synthesis primer-dimer. The fluorescent dye used in the primer-dimer detection probe is detectably distinct from the fluorescent dye used in the adapter-specific probe.

In certain embodiments of the methods provided herein, the primer-dimer detection probe comprises a locked nucleic acid (LNA) probe pair comprising a first LNA probe comprising a fluorescent dye attached to either the 3′- or 5′-end of the first probe and a series of nucleotides that are complementary to the nucleic acid sequence of the sequencing by synthesis primer-dimer, and a second LNA probe comprising a fluorescence quencher attached to either the 3′- or 5′-end of the second probe and a series of nucleotides that are complementary to the nucleic acid sequence of the first LNA probe (e.g., complementary to the first LNA probe). In certain embodiments, the first LNA probe comprises 8-12 nucleotides that are complementary to the nucleic acid sequence of the sequencing by synthesis primer-dimer. The fluorescent dye and quencher used in the primer-dimer detection probe are detectably distinct from the fluorescent dye and quencher used in the adapter-specific probe.

In certain embodiments, the one or more fluorescent dye attached to the nucleic acid-based probes disclosed herein (e.g., the adapter-specific probe and/or the primer-dimer detection probe) is selected from the group consisting of, but not limited to, the group chosen from a pyrene, a xanthene, a cyanine, an indole, a benzofuran, a coumarin, or a borapolyazaindacene. In certain embodiments, the one or more quencher attached to the nucleic acid-based probes disclosed herein (e.g., the adapter-specific probe and/or the primer-dimer detection probe) is selected from the group chosen from BLACK HOLE QUENCHER® dye (Biosearch Technologies Inc., Petaluma, Calif.), an IOWA BLACK® Quencher (Integrated DNA Technologies, Inc., Coralville, Iowa), a QSY® Quencher (Thermo Fisher Scientific, Waltham, Mass.), Dabsyl, Dabcel, a Deep Dark Quencher (Kaneka Eurogentec, S.A., Seraing, Belgium), and an ECLIPSE® Quencher (Glen Research, Sterling, Va.). In certain embodiments, fluorophore-quencher combinations are selected from those listed in Table 1 below.

TABLE 1

| Fluorophore | Quencher | FRET Fluorophore |
|---|---|---|
| Fluorescein | Dabcyl | Tetramethylrhodamine |
| ALEXA | BLACK HOLE | ALEXA |
| FLUOR ® 488* | QUENCHER ® | FLUOR ® 555 |
| | IOWA | |
| | BLACK ® FQ | |
| | QSY 9 | |
| ALEXA | QSY 9 | ALEXA |
| FLUOR ® 555 | | FLUOR ® 647 |

(*ALEXA FLUOR, Thermo Fisher Scientific, Waltham, MA)

Kits

Kits for performing the methods described herein are also provided. As used herein, the term “kit” refers to a packaged set of related components, typically one or more compounds or compositions. The kit may comprise one or more nucleic acid-based probes for quantifying at least one target nucleic acid from a sample. The kit may also include samples containing pre-defined target nucleic acids to be used in control reactions. The kit may also optionally include stock solutions, buffers, enzymes, detectable labels or reagents required for detection, tubes, membranes, and the like that may be used to quantify the target nucleic acid.

In certain embodiments, kits are provided for detecting or quantifying nucleic acids, the kits comprising: one or more adapter-specific probe, a buffer, and instructions for detecting or quantifying nucleic acids according to one or more of the methods described herein. In certain embodiments, the kit comprises two adapter-specific probes. In certain embodiments, the kit further comprises a primer-dimer detection probe.

In certain embodiments of the kits provided herein, the adapter-specific probe comprises a peptide nucleic acid (PNA) oligomer comprising a fluorescent dye attached to the N-terminus of the PNA oligomer, a quencher attached to the C-terminus of the PNA oligomer, and a series of nucleotides that are complementary to a nucleic acid sequence of the sequencing by synthesis adapter. In certain embodiments, the PNA oligomer comprises 8-12 nucleotides that are complementary to a nucleic acid sequence of the sequencing by synthesis adapter. In certain embodiments of the kits provided herein, the adapter-specific probe comprises a molecular beacon comprising a fluorescent dye attached to the 5′-end of the molecular beacon, a quencher attached to the 3′-end of the molecular beacon, and 18 nucleotides in the loop portion that are complementary to a nucleic acid sequence of the sequencing by synthesis adapter. In certain embodiments of the kits provided herein, the adapter-specific probe comprises a single-stranded DNA flare comprising a fluorescent dye covalently attached to one or more nucleotides, and 28 nucleotides that are complementary to a nucleic acid sequence of the sequencing by synthesis adapter. In certain embodiments of the kits provided herein, the adapter-specific probe comprises a locked nucleic acid (LNA) probe pair comprising a first LNA probe comprising a fluorescent dye attached to either the 3′- or 5′-end of the first probe and a series of nucleotides that are complementary to a nucleic acid sequence of the sequencing by synthesis adapter, and a second LNA probe comprising a fluorescence quencher attached to either the 3′- or 5′-end of the second probe and a series of nucleotides that are complementary to the nucleic acid sequence of the first LNA probe (e.g., complementary to the first LNA probe).

In certain embodiments of the kits provided herein, the primer-dimer detection probe comprises a peptide nucleic acid (PNA) oligomer comprising a fluorescent dye attached to the N-terminus of the PNA oligomer, a quencher attached to the C-terminus of the PNA oligomer, and a series of nucleotides that are complementary to a nucleic acid sequence of the sequencing by synthesis primer-dimer. In certain embodiment, the PNA oligomer comprises 8-12 nucleotides that are complementary to a nucleic acid sequence of the sequencing by synthesis primer-dimer. In certain embodiments of the methods provided herein, the primer-dimer detection probe comprises a molecular beacon comprising a fluorescent dye attached to the 5′-end, a quencher attached to the 3′-end, and 18 nucleotides in the loop portion that are complementary to a nucleic acid sequence of the sequencing by synthesis primer-dimer. In certain embodiments of the methods provided herein, the primer-dimer detection probe comprises a DNA flare probe comprising a fluorescent dye covalently attached to one or more nucleotides, and 28 nucleotides that are complementary to a nucleic acid sequence of the sequencing by synthesis primer-dimer. In certain embodiments of the methods provided herein, the adapter-specific probe comprises a locked nucleic acid (LNA) probe pair comprising a first LNA probe comprising a fluorescent dye attached to either the 3′- or 5′-end of the first probe and a series of nucleotides that are complementary to a nucleic acid sequence of the sequencing by synthesis primer-dimer, and a second LNA probe comprising a fluorescence quencher attached to either the 3′- or 5′-end of the second probe and a series of nucleotides that are complementary to the nucleic acid sequence of the first LNA probe (e.g., complementary to the first LNA probe). The fluorescent dye or the fluorescent dye-quencher pair used in any of the primer-dimer detection probes is detectably distinct from the fluorescent dye and quencher used in the adapter-specific probe.

Preferred kits may comprise one or more containers, such as vials, tubes and the like, configured to contain the reagents used in the methods described herein, including nucleic acid-based probes, buffers, and the like, and optionally may contain instructions or protocols for using such reagents according to the methods disclosed herein. The kits described herein may comprise one or more components selected from the group consisting of one or more oligonucleotides described herein, including but not limited to, one or more nucleic acid-based probe, such as an adapter-specific probe or a primer-dimer detection probe.

Such kits can be prepared from readily available materials and reagents and can come in a variety of embodiments. The contents of the kit will depend on the design of the method or assay protocol for detection or measurement. Typically, instructions include a tangible expression describing the reagent concentration or at least one assay method parameter such as the relative amounts of reagent and sample to be added together, maintenance time periods for reagent/sample admixtures, temperature, buffer conditions and the like to allow the user to carry out any one of the methods or preparations described above. In one aspect, the kit is formulated to facilitate the high-throughput screening of multiple samples, such as may be accomplished using automated methods.

Thus, a kit for detecting nucleic acid in a sample may comprise one or more of an adapter-specific probe as described above. The kit may further comprise one or more of a primer-dimer detection probe. The kit may further include instructions for performing one or more of the above disclosed methods, including the detection and/or quantitation of nucleic acid, such as DNA to be used in next generation sequencing applications.

The kit may optionally further include one or more of the following; sample preparation reagents, a buffering agent, nucleic acid standards, an aqueous nucleic acid reporter molecule dilution buffer or an organic solvent.

### Example 1: Exemplary Method for Quantifying DNA Samples to be Used in Next Generation Sequencing (Illumina Platform)

In the method described in this example, a unique nucleic acid-based probe (e.g., an adapter-specific probe and/or a primer-dimer detection probe) is used based on a peptide nucleic acid (PNA) oligomer, a molecular beacon probe, a DNA flare probe or a locked nucleic acid (LNA) probe pair, and the target DNA sequence comprises the conserved Illumina adapter sequence. The method and materials used in this example are detailed below. The nucleic acid-based probe can be any of the PNA, molecular beacon, DNA flare or LNA type, but the type of probe must be the same for the standards and the sample.

Required Materials:
dsDNA sample with Illumina adapter modification
Nucleic acid-based probe, which contains equal amounts of the adapter-specific probe and the primer-dimer detection probe (PNA, molecular beacon, DNA flare or LNA-based probe)
Hot plate or heating apparatus
Microfuge tube or 96 well-plate
Buffer

User Workflow:
Step 1. Label a 0.5 mL microfuge tube as “Sample” and combine:
   a. 5 μL nucleic acid-based probe (contains equal amounts of the adapter-specific probe and primer-dimer detection probe)
   b. 2 μL DNA sample
   c. 193 μL of buffer
Step 2. Repeat Step 1 for all samples and replicates.
Step 3. Label a 0.5 mL microfuge tube as “Standard #1” and combine:
   a. 5 μL nucleic acid-based probe (contains equal amounts of the adapter-specific probe and primer-dimer detection probe)
   b. 10 μL of Standard #1
   c. 195 μL of buffer
Step 4. Label a 0.5 mL microfuge tube as “Standard #2” and combine:
   a. 5 μL nucleic acid-based probe (contains equal amounts of the adapter-specific probe and primer-dimer detection probe)
   b. 10 μL of Standard #2
   c. 195 μL of buffer
Step 5. Heat all samples sample to 90° C. for 5 minutes to allow for full denaturation of the sample dsDNA.
Step 6. Cool the samples to 70° C. for 2 minutes to allow the probe to recognize the target sequence.
Step 7. Allow the samples to cool to room temperature.
Step 8. Measure the fluorescence of the samples:
   a. Using a QUBIT™ Fluorometer (Thermo Fisher Scientific, Waltham, Mass.)
      i. Read the samples labeled “Standard #1” and “Standard #2”. This calibrates the instrument and allows for proper interpretation of the unknown sample.
      ii. Measure each unknown sample. The instrument will report concentrations for samples that contains the adapter sequence and primer-dimers using molarity based units (molecules of DNA in the sample containing these specific motifs relative to volume).
      iii. Repeat for all samples
   b. Using a plate reader
      i. Transfer the contents of each sample tube to a 96-well microplate.
      ii. Using the proper excitation and emission for both the adapter-specific probe and the primer-dimer detection probe, read the fluorescence of the samples.
      iii. Using the values from Standard #1 and Standard #2, prepare a linear regression where RFU=Concentration of sample*m+b for each probe present.
      iv. Rearrange the equation so that Concentration of sample=(RFU−b)/m, and solve the equation using each unknown samples measured RFU value.

The QUBIT™ Fluorometer instrument measures the fluorescence of the two nucleic acid-based probes (the adapter-specific probe and the primer-dimer detection probe), and using the standard curve, provides the user (or for a plate reader allow the user to calculate) with molar concentrations of the adapter modified DNA and primer-dimers.

### Example 2—Exemplary Method for Quantifying DNA Samples to be Used in Next Generation Sequencing (Illumina Platform)

In the method described in this example, a unique nucleic acid-based probe (e.g., an adapter-specific probe and/or a primer-dimer detection probe) is used based on a peptide nucleic acid (PNA) oligomer, a molecular beacon probe, a DNA flare probe or a locked nucleic acid (LNA) probe pair, and the target DNA sequence comprises the conserved Illumina adapter sequence. The method and materials used in this example are detailed below. The nucleic acid-based probe can be any of the PNA, molecular beacon, DNA flare or LNA type, but the type of probe must be the same for the standards and the sample.

Required Materials:
dsDNA sample with Illumina adapter modification
Nucleic acid-based probe, which contains equal amounts of the adapter-specific probe and the primer-dimer detection probe (PNA, molecular beacon, DNA flare or LNA-based probe)
Microfuge tube or 96 well-plate
Buffer

User Workflow:
Step 1. Label a 0.5 mL microfuge tube as “Sample” and combine:
   a. 5 μL nucleic acid-based probe (contains equal amounts of the adapter-specific probe and primer-dimer detection probe)
   b. 2 μL DNA sample
   c. 193 μL of buffer
Step 2. Repeat Step 1 for all samples and replicates.
Step 3. Label a 0.5 mL microfuge tube as “Standard #1” and combine:
   a. 5 μL nucleic acid-based probe (contains equal amounts of the adapter-specific probe and primer-dimer detection probe)
   b. 10 μL of Standard #1
   c. 195 μL of buffer
Step 4. Label a 0.5 mL microfuge tube as “Standard #2” and combine:
   a. 5 μL nucleic acid-based probe (contains equal amounts of all probes containing in the assay)
   b. 10 μL of Standard #2
   c. 195 μL of buffer
Step 5. Mix samples and allow to incubate.
Step 6. Measure the fluorescence of the samples:
   a. Using a QUBIT™ Fluorometer (Thermo Fisher Scientific, Waltham, Mass.)
      i. Read the samples labeled “Standard #1” and “Standard #2”. This calibrates the instrument and allows for proper interpretation of the unknown sample.
      ii. For systems containing multiple probes, a Standard #2 will be required for each additional probe.
      iii. Measure each unknown sample. The instrument will report concentrations for samples that contains the adapter sequence and primer-dimers using molarity based units (molecules of DNA in the sample containing these specific motifs relative to volume).
      iv. Repeat for all samples
   b. Using a plate reader
      i. Transfer the contents of each sample tube to a 96-well microplate.
      ii. Using the proper excitation and emission for both the adapter-specific probe and the primer-dimer detection probe, read the fluorescence of the samples.
      iii. Using the values from Standard #1 and Standard #2, prepare a linear regression where RFU=Concentration of sample*m+b for all probes present.
      iv. Rearrange the equation so that Concentration of sample=(RFU-b)/m, and solve the equation using each unknown samples measured RFU value.

The QUBIT™ Fluorometer instrument measures the fluorescence of the two nucleic acid-based probes (the adapter-specific probe and the primer-dimer detection probe), and using the standard curve, provides the user (or for a plate reader allow the user to calculate) with molar concentrations of the adapter modified DNA and primer-dimers.

### Example 3—DNA Flare Probe Construction and Analysis

Oligonucleotides for use as DNA flare probes were constructed using phosphoramidite chemistry using FAM-dT as the fluorescent dye. The nucleotide sequence and placement of FAM-dT in exemplary DNA flare probes are listed in Tables 2 and 3.

TABLE 2

| | SEQ | |
|---|---|---|
| Probe name | ID NO: | Sequence (5 = amine, 4 = fluorescein-dT) |
| 5FAM12-63 | 1 | 5GAC4GACTGACTGAC4GACTGACTGAC4GACTGACTGAC4GACTGACTGA |
| | | C4GACTGACTGAC |
| Comp-63 | 2 | GTCAGTCAGTCAGTCAGTCAGTCAGTCAGTCAGTCAGTCAGTCAGTCAGT |
| | | CAGTCAGTCAGTC |
| 4FAM12-51 | 3 | 5GAC4GACTGACTGAC4GACTGACTGAC4GACTGACTGAC4GACTGACTGA |
| | | C |
| Comp-51 | 4 | GTCAGTCAGTCAGTCAGTCAGTCAGTCAGTCAGTCAGTCAGTCAGTCAGT |
| | | C |
| 3FAM-39 | 5 | 5GAC4GACTGACTGAC4GACTGACTGAC4GACTGACTGAC |
| Comp-39 | 6 | GTCAGTCAGTCAGTCAGTCAGTCAGTCAGTCAGTCAGTC |
| 2FAM-27 | 7 | 5GAC4GACTGACTGAC4GACTGACTGAC |
| Comp-27 | 8 | GTCAGTCAGTCAGTCAGTCAGTCAGTC |
| 1FAM-15 | 9 | 5GAC4GACTGACTGAC |
| Comp-15 | 10 | GTCAGTCAGTCAGTC |
| 6FAM12-71 | 11 | 5GAC4GACTGACTGAC4GACTGACTGAC4GACTGACTGAC4GACTGACTGA |
| | | C4GACTGACTGAC4GACTGAC |
| 3FAM32-71 | 12 | 5GAC4GACTGACTGACTGACTGACTGACTGACTGAC4GACTGACTGACTG |
| | | ACTGACTGACTGACTGAC4GAC |
| 5FAM16-71 | 13 | 5GAC4GACTGACTGACTGAC4GACTGACTGACTGAC4GACTGACTGACTGA |
| | | C4GACTGACTGACTGAC4GAC |
| 9FAM8-71 | 14 | 5GAC4GACTGAC4GACTGAC4GACTGAC4GACTGAC4GACTGAC4GACTGA |
| | | C4GACTGAC4GACTGAC4GAC |
| Comp-71 | 15 | GTCAGTCAGTCAGTCAGTCAGTCAGTCAGTCAGTCAGTCAGTCAGTCAGT |
| | | CAGTCAGTCAGTCAGTCAGTC |

TABLE 3

| | SEQ | |
|---|---|---|
| Probe Name | ID NO: | Sequence (5 = amine, 2 = fluorescein-dT) |
| 6FAM3-23 | 16 | 5GAC2GA2TG2CT2AC2GA2TGAC |
| Comp-23 | 17 | GTCAATCAGTAAGACAATCAGTC |
| 6FAM-4-28 | 18 | 5GAC2GAC2GAC2GAC2GAC2GAC2GACT |
| Comp-28 | 19 | AGTCAGTCAGTCAGTCAGTCAGTCAGTC |
| 6FAM5 -33 | 20 | 5GAC2GACT2ACTG2CTGA2TGAC2GACT2ACTG |
| Comp-33 | 21 | CAGTAAGTCAGTCAATCAGACAGTAAGTCAGTC |
| 6FAM6-38 | 22 | 5GAC2GATCG2CTGAC2GACTG2CTGAC2GACTG2CTGA |
| Comp-38 | 23 | TCAGACAGTCAGTCAGACAGTCAGTCAGACGATCAGTC |
| 6FAM7-43 | 24 | 5GAC2GACTGA2TGACTG2CTGACT2ACTGAC2GACTGA2TGAC |
| Comp-43 | 25 | GTCAATCAGTCAGTCAGTAAGTCAGACAGTCAATCAGTCAGTC |
| 6FAM8-48 | 26 | 5GAC2GACTGAC2GACTGAC2GACTGAC2GACTGAC2GACTGAC2GACT |
| Comp-48 | 27 | AGTCAGTCAGTCAGTCAGTCAGTCAGTCAGTCAGTCAGTCAGTCAGTC |
| 6FAM9-53 | 28 | 5GAC2GACTGACT2ACTGACTG2CTGACTGA2TGACTGAC2GACTGACT2AC |
| | | TG |
| Comp-53 | 29 | CAGTAAGTCAGTCAGTCAGTCAATCAGTCAGACAGTCAGTAAGTCAGTCA |
| | | GTC |
| 6FAM10-58 | 30 | 5GAC2GACTGACTG2CTGACTGAC2GACTGACTG2CTGACTGAC2GACTGAC |
| | | TG2CTGA |
| Comp-58 | 31 | TCAGACAGTCAGTCAGTCAGTCAGACAGTCAGTCAGTCAGTCAGACAGTC |
| | | AGTCAGTC |
| 6FAM11-63 | 32 | 5GAC2GACTGACTGA2TGACTGACTG2CTGACTGACT2ACTGACTGAC2GAC |
| | | TGACTGA2TGAC |
| Comp-63 | 33 | GTCAATCAGTCAGTCAGTCAGTCAGTAAGTCAGTCAGACAGTCAGTCAAT |
| | | CAGTCAGTCAGTC |

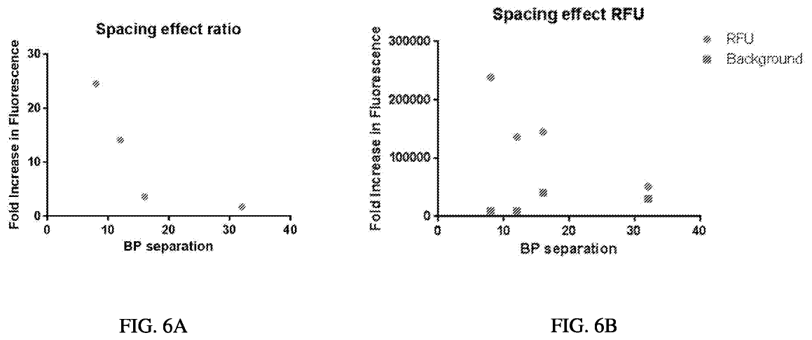

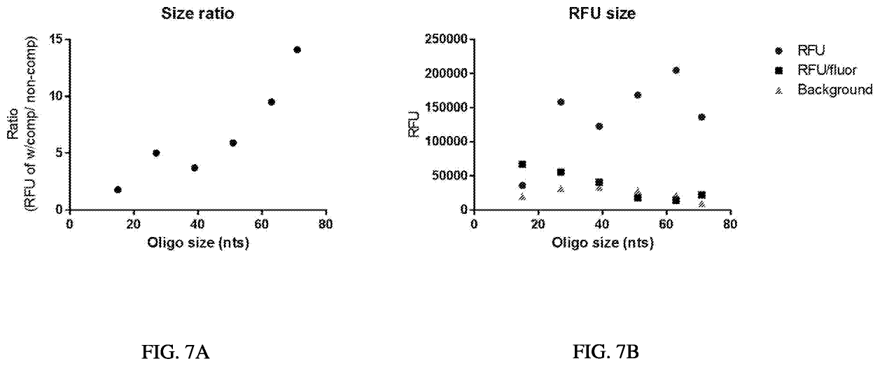

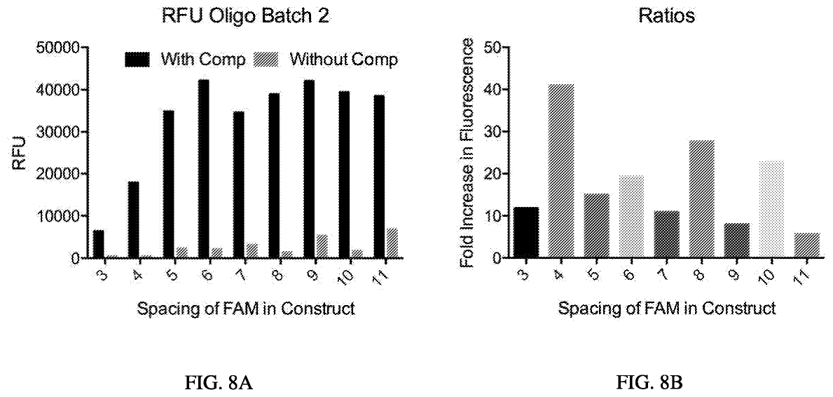

In Tables 2 and 3 each sequence was given a name based on the number of FAMs, the spacing, and the overall length (e.g. 5FAM12-63 means five FAM molecules, spaced 12 bases apart in a 63 nucleotide (nt) long sequence). The unmodified reverse complement oligonucleotides were created to mimic complementation in the target system and are labeled comp-x, e.g. Comp-63. The data shown in FIGS. 6A through 8B were obtained by assessing the fluorescence of the single stranded probe (quenched state) and then measuring the fluorescence after the probe was annealed to its complement (unquenched state). The ratio of the two values yielded the fold increase in fluorescence. In Table 2, all three parameters (oligonucleotide length, number of fluorescent dye molecules and spacing between dye molecules) were altered. The only two probes which were not fluorogenic were an oligonucleotide with a single fluorescent dye and 3FAM-32 which showed if the fluorescent dyes were spaced far apart then quenching occurred and was not reversible (See FIGS. 6A and 6B). In Table 3, the fluorescent dye molecule count was held steady at 6 and the spacing between fluorescent dye molecules was sequentially reduced from 11 to 3 bases, which also reduced the overall length of the oligonucleotides. However, the overall length of the oligonucleotide did not appear to have much of an effect on the fluorescence of the DNA flare (See FIGS. 7A and 7B). Most of the fluorescence was lost when the fluorescent dye molecules were spaced 3 bases apart and half of the fluorescent signal was lost at 4 bases separation (see FIG. 8A), but the noise was reduced substantially at 4 bases separation which led to a fold fluorescence increase of over 40 for this construct (see, FIG. 8B).

### Example 4—Detection and Analysis Using a One Probe System

A single fluorogenic probe specific to the 3′-end or the 5′-end of the Illumina adapter sequence is used to quantify the degree of DNA sample that has been modified to contain the sequencing by synthesis adapter sequence. The probe itself can be a peptide nucleic acid (PNA) oligomer, a molecular beacon probe (DNA hairpin), a DNA flare probe or a locked nucleic acid (LNA) probe pair, and the target DNA sequence comprises the conserved Illumina adapter sequence. Upon recognition of the target sequence, the probe emits a detectable signal that is proportional to the amount of target sequence present in the sample. In the absence of the target sequence, the probe emits a negligible signal.

### Example 5—Detection and Analysis Using a Two Probe System

Two fluorescent probes, one specific to the 3′-end or the 5′-end of the sequencing by synthesis adapter sequence and the second specific to the primer-dimer sequence, are used to quantify the DNA sample that has been modified to contain the sequencing by synthesis adapter sequence. The probes can be peptide nucleic acid (PNA) oligomers, molecular beacon probes (DNA hairpin), DNA flare probes, locked nucleic acid (LNA) probe pairs, or a combination thereof. The target DNA sequence comprises the conserved sequencing by synthesis adapter sequence. Upon recognition of the target sequence, the probes emit a detectable signal that is proportional to the amount of target sequence present in the sample. In the absence of the target sequence, the probes emit a negligible signal. This approach enables a direct quantification of the degree of single adapter modification as well as the detection of the primer-dimer sequence.

Two fluorescent probes, one specific to the 3′-end of the sequencing by synthesis adapter sequence and the second specific to the 5′-end of the sequencing by synthesis adapter sequence, are used to quantify the DNA sample that has been modified to contain the sequencing by synthesis adapter sequence. The probes can be peptide nucleic acid (PNA) oligomers, molecular beacon probes (DNA hairpin), DNA flare probes, locked nucleic acid (LNA) probe pairs, or a combination thereof. The target DNA sequence comprises the conserved Illumina adapter sequence. Upon recognition of the target sequence, the probes emit a detectable signal that is proportional to the amount of target sequence present in the sample. In the absence of the target sequence, the probes emit a negligible signal. This approach enables a direct quantification of the degree of full adapter modification.

### Example 6—Detection and Analysis Using a Three Part Probe System

Three fluorescent probes, one specific to the 3′-end of the sequencing by synthesis adapter sequence, a second specific to the 5′-end of the sequencing by synthesis adapter sequence, and the third specific to the Illumina primer-dimer sequence, are used to quantify the DNA sample that has been modified to contain the Illumina adaptor sequence. The probes can be peptide nucleic acid (PNA) oligomers, molecular beacon probes (DNA hairpin), DNA flare probes, locked nucleic acid (LNA) probe pairs, or a combination thereof. The target DNA sequence comprises the conserved sequencing by synthesis adapter sequence. Upon recognition of the target sequence, the probes emit a detectable signal that is proportional to the amount of target sequence present in the sample. In the absence of the target sequence, the probes emit a negligible signal. This approach enables a direct quantification of the degree of full adapter modification as well as the detection of the primer-dimer sequence.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detecting a nucleic acid in a sample, the method comprising:
a) combining a nucleic acid probe with the sample to prepare a probe-nucleic acid mixture;
b) incubating the probe-nucleic acid mixture for a sufficient amount of time for the nucleic acid probe to hybridize to the nucleic acid in the sample to form a probe-nucleic acid complex;
c) illuminating the probe-nucleic acid complex with an appropriate wavelength of light to generate a detectable optical response; and
d) detecting the detectable optical response thereby detecting the nucleic acid.

2. A method for quantifying a nucleic acid in a sample, the method comprising:
a) combining a nucleic acid probe with the sample to prepare a probe-nucleic acid mixture;
b) incubating the probe-nucleic acid mixture for a sufficient amount of time for the nucleic acid probe to hybridize to the nucleic acid in the sample to form a probe-nucleic acid complex;
c) illuminating the probe-nucleic acid complex with an appropriate wavelength of light to generate a detectable optical response;
d) detecting the detectable optical response; and
e) comparing the detectable optical response with a known quantity of nucleic acid thereby quantifying the nucleic acid in the sample.

3. A method of detecting a target nucleic acid in a sample, the method comprising:
a) providing a sample containing a target nucleic acid comprising at least one sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence; and
c) incubating the target-probe mixture for a sufficient amount of time to allow the adapter-specific probe to hybridize to the target nucleic acid to form a probe-target complex.

4. The method of claim 3 further comprising:
d) illuminating the probe-target complex with an appropriate wavelength of light to generate a detectable optical response; and
e) detecting the detectable optical response thereby detecting the target nucleic acid in the sample.

5. The method of claim 3 or 4, wherein the adapter-specific probe is complementary to a 3′-end region or a 5′-end region of the sequencing by synthesis adapter sequence.

6. A method of detecting a target nucleic acid in a sample is provided, the method comprising:
a) providing a sample containing a target-probe mixture, wherein the target-probe mixture comprises i) a target nucleic acid comprising at least one sequencing by synthesis adapter sequence, and ii) an adapter-specific probe comprising a nucleic acid sequence that is complementary to the sequencing by synthesis adapter sequence;
b) illuminating the target-probe mixture with an appropriate wavelength of light to generate a detectable optical response; and
c) detecting the detectable optical response thereby detecting the target nucleic acid in the sample.

7. A method of detecting a target nucleic acid in a sample, the method comprising:
a) providing a sample containing one or more target-probe mixtures, wherein the one or more target-probe mixtures comprises i) a target nucleic acid comprising at least one sequencing by synthesis adapter sequence, and ii) one or more adapter-specific probe comprising a nucleic acid sequence that is complementary to the sequencing by synthesis adapter sequence;
b) illuminating the one or more target-probe mixture with an appropriate wavelength of light to generate a detectable optical response; and
c) detecting the detectable optical response thereby detecting the target nucleic acid in the sample.

8. The method of claim 7, wherein the sample contains two target-probe mixtures.

9. The method of claim 7 or 8, wherein the adapter-specific probe is complementary to a 3′-end region or a 5′-end region of the sequencing by synthesis adapter sequence.

10. A method of detecting a target nucleic acid in a sample, the method comprising:
a) providing a sample containing a target nucleic acid comprising at least one sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence; and
d) incubating the first target-probe mixture and the second target-probe mixture for a sufficient amount of time to allow the first adapter-specific probe and the second adapter-specific probe to hybridize to the target nucleic acid to form a first probe-target complex and a second probe-target complex;
wherein the first adapter-specific probe and the second adapter-specific probe are detectably distinct.

11. The method of claim 10, further comprising:
e) illuminating the first probe-target complex and the second probe-target complex with an appropriate wavelength of light to generate a first detectable optical response and a second detectable optical response; and
f) detecting the first detectable optical response and the second detectable optical response thereby detecting the target nucleic acid in the sample.

12. The method of claim 10 or 11, wherein:
the first region of the sequencing by synthesis adapter sequence is a 3′-end region and the second region of the sequencing by synthesis adapter sequence is a 5′-end region; or
the first region of the sequencing by synthesis adapter sequence is a 5′-end region and the second region of the sequencing by synthesis adapter sequence is a 3′-end region.

13. The method of claim 10, 11 or 12, wherein:
step (b) and step (c) are performed simultaneously; or
step (b) and step (c) are performed sequentially.

14. A method of quantifying a target nucleic acid in a sample, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence; and
c) incubating the target-probe mixture for a sufficient amount of time to allow the adapter-specific probe to hybridize to the target nucleic acid to form a probe-target complex.

15. The method of claim 14, further comprising:
d) illuminating the probe-target complex with an appropriate wavelength of light to generate a detectable optical response;
e) detecting the detectable optical response; and
f) comparing the detectable optical response with a known quantity of nucleic acid thereby quantifying the target nucleic acid in the sample.

16. The method of claim 14 or 15, wherein the adapter-specific probe is complementary to a 3′-end region or a 5′-end region of the sequencing by synthesis adapter sequence.

17. A method of quantifying a target nucleic acid in a sample, the method comprising:
a) providing a sample containing a target nucleic acid comprising at least one sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence; and
d) incubating the first target-probe mixture and the second target probe mixture for a sufficient amount of time to allow the first adapter-specific probe and the second adapter-specific probe to hybridize to the target nucleic acid to form a first probe-target complex and a second probe-target complex;
wherein the first adapter-specific probe and the second adapter-specific probe are detectably distinct.

18. The method of claim 17, further comprising:
e) illuminating the first probe-target complex and the second probe-target complex with an appropriate wavelength of light to generate a first detectable optical response and a second detectable optical response; and
f) detecting the first detectable optical response and the second detectable optical response thereby detecting the target nucleic acid in the sample;
g) comparing the first detectable optical response and the second detectable optical response with a known quantity of nucleic acid thereby quantifying the target nucleic acid in the sample.

19. The method of claim 17, wherein:
the first region of the sequencing by synthesis adapter sequence is a 3′-end region and the second region of the sequencing by synthesis adapter sequence is a 5′-end region; or
the first region of the sequencing by synthesis adapter sequence is a 5′-end region and the second region of the sequencing by synthesis adapter sequence is a 3′-end region.

20. The method of claim 17, 18 or 19, wherein:
step (b) and step (c) are performed simultaneously; or
step (b) and step (c) are performed sequentially.

21. A method of quantifying a target nucleic acid in a sample, the method comprising:
a) providing a sample containing a target-probe mixture, wherein the target-probe mixture comprises i) a target nucleic acid comprising a sequencing by synthesis adapter sequence, and ii) an adapter-specific probe comprising a nucleic acid sequence that is complementary to the sequencing by synthesis adapter sequence;
b) illuminating the target-probe mixture with an appropriate wavelength of light to generate a detectable optical response;
c) detecting the detectable optical response; and
d) comparing the detectable optical response with a known quantity of nucleic acid thereby quantifying the target nucleic acid in the sample.

22. A method of quantifying a target nucleic acid in a sample, the method comprising:
a) providing a sample containing one or more target-probe mixtures, wherein the one or more target-probe mixtures comprises i) a target nucleic acid comprising at least one sequencing by synthesis adapter sequence, and ii) one or more adapter-specific probes comprising a nucleic acid sequence that is complementary to the sequencing by synthesis adapter sequence;
b) illuminating the one or more target-probe mixture with an appropriate wavelength of light to generate a detectable optical response; and
c) detecting the detectable optical response thereby detecting the target nucleic acid in the sample.

23. The method of claim 22, wherein the sample contains two target-probe mixtures.

24. The method of claim 22 or 23, wherein the adapter-specific probe is complementary to a 3′-end region or a 5′-end region of the sequencing by synthesis adapter sequence.

25. A method of detecting a target nucleic acid in a sample, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence;
c) heating the target-probe mixture for a sufficient amount of time to allow the target nucleic acid to denature;
d) cooling the target-probe mixture for a sufficient amount of time to allow the adapter-specific probe to hybridize to the target nucleic acid to create a probe-target complex;
e) illuminating the probe-target complex with an appropriate wavelength of light to generate a detectable optical response; and
f) detecting the detectable optical response thereby detecting the target nucleic acid in the sample.

26. The method of claim 25, wherein the first adapter-specific probe is complementary to a 3′-end region or a 5′-end region of the sequencing by synthesis adapter sequence.

27. A method of detecting a target nucleic acid in a sample, the method comprising:
a) providing a sample containing a target nucleic acid comprising a sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence;
d) heating the first target-probe mixture and the second target-probe mixture for a sufficient amount of time to allow the target nucleic acid to denature;
e) cooling the first target-probe mixture and the second target-probe mixture for a sufficient amount of time to allow the first adapter-specific probe and the second adapter-specific probe to hybridize to the target nucleic acid to create a first probe-target complex and a second probe-target complex;
f) illuminating the first probe-target complex and the second probe-target complex with an appropriate wavelength of light to generate a first detectable optical response and a second detectable optical response; and
g) detecting the first detectable optical response and the second detectable optical response thereby detecting the target nucleic acid in the sample,
wherein the first adapter-specific probe and the second adapter-specific probe are detectably distinct.

28. The method of claim 27, wherein:
the first region of the sequencing by synthesis adapter sequence is a 3′-end region and the second region of the sequencing by synthesis adapter sequence is a 5′-end region; or
the first region of the sequencing by synthesis adapter sequence is a 5′-end region and the second region of the sequencing by synthesis adapter is a 3′-end region.

29. The method of claim 27 or 28, wherein:
step (b) and step (c) are performed simultaneously; or
step (b) and step (c) are performed sequentially.

30. A method of quantifying a target nucleic acid in a sample, the method comprising:
a) providing a sample containing a target nucleic acid comprising a sequencing by synthesis adapter sequence;
b) combining the sample with an adapter-specific probe to create a target-probe mixture, wherein the adapter-specific probe comprises a nucleic acid sequence that is complementary to the sequencing by synthesis adapter sequence;
c) heating the target-probe mixture for a sufficient amount of time to allow the target nucleic acid to denature;
d) cooling the target-probe mixture for a sufficient amount of time to allow the adapter-specific probe to hybridize to the target nucleic acid to create a probe-target complex;
e) illuminating the probe-target complex with an appropriate wavelength of light to generate a detectable optical response;
f) detecting the detectable optical response; and
g) comparing the detectable optical response with a known quantity of nucleic acid thereby quantifying the target nucleic acid in the sample.

31. The method of claim 30, wherein the adapter-specific probe is complementary to a 3′-end region or a 5′-end region of the sequencing by synthesis adapter sequence.

32. A method of quantifying a target nucleic acid in a sample, the method comprising:
a) providing a sample containing a target nucleic acid comprising a sequencing by synthesis adapter sequence;
b) combining the sample with a first adapter-specific probe to create a first target-probe mixture, wherein the first adapter-specific probe comprises a nucleic acid sequence that is complementary to a first region of the sequencing by synthesis adapter sequence;
c) combining the sample with a second adapter-specific probe to create a second target-probe mixture, wherein the second adapter-specific probe comprises a nucleic acid sequence that is complementary to a second region of the sequencing by synthesis adapter sequence;
d) heating the first target-probe mixture and the second target-probe mixture for a sufficient amount of time to allow the target nucleic acid to denature;
e) cooling the first target-probe mixture and the second target-probe mixture for a sufficient amount of time to allow the first adapter-specific probe and the second adapter-specific probe to hybridize to the target nucleic acid to create a first probe-target complex and a second target-probe complex;
f) illuminating the first probe-target complex and the second target-probe complex with an appropriate wavelength of light to generate a first detectable optical response and a second detectable optical response;
g) detecting the first detectable optical response and the second detectable optical response; and
h) comparing the first detectable optical response and the second detectable optical response with a known quantity of nucleic acid thereby quantifying the target nucleic acid in the sample,
wherein the first adapter-specific probe and the second adapter-specific probe are detectably distinct.

33. The method of claim 32, wherein:
the first region of the sequencing by synthesis adapter sequence is a 3′-end region and the second region of the sequencing by synthesis adapter sequence is a 5′-end region; or
the first region of the sequencing by synthesis adapter sequence is a 5′-end region and the second region of the sequencing by synthesis adapter sequence is a 3′-end region.

34. The method of claim 32 or 33, wherein:
step (b) and step (c) are performed simultaneously; or
step (b) and step (c) are performed sequentially.

35. The method according to any one of the preceding claims, wherein the target nucleic acid is double-stranded DNA.

36. The method according to any one of claims 3 to 35, wherein the adapter-specific probe comprises:
an oligonucleotide sequence that is complementary to the sequencing by synthesis adapter sequence, or a portion thereof; and
a fluorescent dye.

37. The method according to claim 36, wherein the oligonucleotide sequence is complementary to a 3′-end region or a 5′-end region of the sequencing by synthesis adapter sequence, or a portion thereof.

38. The method according to claim 36, wherein the adapter-specific probe further comprises a quencher.

39. The method according to any one of claims 3 to 38, wherein the adapter-specific probe comprises a peptide nucleic acid (PNA) oligomer probe, a molecular beacon probe, a DNA flare probe or a locked nucleic acid (LNA) probe pair.

40. The method according to any one of the preceding claims, wherein the adapter-specific probe comprises a peptide nucleic acid oligomer (PNA) comprising a fluorescent dye, a quencher, and a series of nucleotides that are complementary to the sequencing by synthesis adapter.

41. The method according to any one of claims 3 to 39, wherein the adapter-specific probe comprises a molecular beacon probe comprising a fluorescent dye, a quencher, and 18 nucleotides in the loop portion that are complementary to the sequencing by synthesis adapter.

42. The method according to any one of claims 3 to 39, wherein the adapter-specific probe comprises a DNA flare probe comprising a fluorescent dye covalently attached to one or more nucleotides, and 28 nucleotides that are complementary to the sequencing by synthesis adapter.

43. The method according to any one of claims 3 to 39, wherein the adapter-specific probe comprises a locked nucleic acid (LNA) probe pair comprising:
a first LNA probe comprising a fluorescent dye and a series of nucleotides that are complementary to the sequencing by synthesis adapter; and
a second LNA probe comprising a fluorescence quencher and a series of nucleotides that are complementary to the first LNA probe.

44. The method according to any one of claims 3 to 43, wherein the method further comprises adding a primer-dimer detection probe to the target-probe mixture, wherein the primer-dimer detection probe has a detectable optical response that is distinguishable from the detectable optical response of the adapter-specific probe.

45. The method according to any one of claims 36-44, wherein the fluorescent dye is a pyrene, a xanthene, a cyanine, an indole, a benzofuran, a coumarin, or a borapolyazaindacene.

46. The method according to any one of claims 38-45, wherein the quencher is BLACK HOLE QUENCHER® dye, an IOWA BLACK® Quencher, a QSY® Quencher, Dabsyl, Dabcel, a Deep Dark Quencher, and an ECLIPSE® Quencher.

47. The method according to any one of the preceding claims, wherein the detecting step is performed by fluorimetry.

48. The method according to any one of the preceding claims, wherein the sample is in a microfuge tube or a multi-well plate.

49. The method according to any one of claims 3 to 48, wherein the sequencing by synthesis adapter is a TRUSEQ® Universal Adapter or a TRUSEQ® Indexed Adapter.

50. An adapter-specific probe comprising:
an oligonucleotide sequence that is complementary to a nucleic acid sequence of a sequencing by synthesis adapter, or a portion thereof; and
a fluorescent dye.

51. The adapter-specific probe according to claim 50, wherein the oligonucleotide sequence is complementary to a 3′-end region or a 5′-end region of the sequencing by synthesis adapter, or a portion thereof.

52. The adapter-specific probe according to claim 50, further comprising a quencher.

53. The adapter-specific probe according to claim 50, wherein the adapter-specific probe comprises a peptide nucleic acid (PNA) oligomer probe, a molecular beacon probe, a DNA flare probe or a locked nucleic acid (LNA) probe pair.

54. The adapter-specific probe according to claim 53, wherein the peptide nucleic acid (PNA) oligomer probe comprises a fluorescent dye, a fluorescence quencher, and a series of nucleotides that are complementary to the nucleic acid sequence of the sequencing by synthesis adapter.

55. The adapter-specific probe according to claim 53, wherein the molecular beacon probe comprises a fluorescent dye, a fluorescence quencher and 18 nucleotides that are complementary to the nucleic acid sequence of the sequencing by synthesis adapter.

56. The adapter-specific probe according to claim 53, wherein the DNA flare probe comprises a fluorescent dye covalently attached to one or more nucleotides, and 28 nucleotides that are complementary to the nucleic acid sequence of the sequencing by synthesis adapter.

57. The adapter-specific probe according to claim 53, wherein the locked nucleic acid (LNA) probe pair comprises:
a first LNA probe comprising a fluorescent dye and a series of nucleotides that are complementary to the nucleic acid sequence of the sequencing by synthesis adapter; and
a second LNA probe comprising a fluorescence quencher and a series of nucleotides that are complementary to the first LNA probe.

58. The adapter-specific probe according to any of claims 50 to 57, wherein the fluorescent dye is a pyrene, a xanthene, a cyanine, an indole, a benzofuran, a coumarin, or a borapolyazaindacene.

59. The adapter-specific probe according to any of claims 51 to 58, wherein the quencher is a BLACK HOLE QUENCHER® dye, an IOWA BLACK® Quencher, a QSY® Quencher, Dabsyl, Dabcel, a Deep Dark Quencher, and an ECLIPSE® Quencher.

60. A primer-dimer detection probe comprising:
an oligonucleotide sequence that is complementary to a nucleic acid sequence of a sequencing by synthesis primer-dimer; and
a fluorescent dye.

61. The primer-dimer detection probe according to claim 60, further comprising a quencher.

62. The primer-dimer detection probe according to claim 60, wherein the primer-dimer detection probe comprises a peptide nucleic acid (PNA) oligomer probe, a molecular beacon probe, a DNA flare probe or a locked nucleic acid (LNA) probe pair.

63. The primer-dimer detection probe according to claim 62, wherein the peptide nucleic acid (PNA) oligomer probe comprises a fluorescent dye, a quencher, and a series of nucleotides that are complementary to the nucleic acid sequence of the sequencing by synthesis primer-dimer.

64. The primer-dimer detection probe according to claim 62, wherein the molecular beacon probe comprises a fluorescent dye, a quencher, and 18 nucleotides that are complementary to the nucleic acid sequence of the sequencing by synthesis primer-dimer.

65. The primer-dimer detection probe according to claim 62, wherein the DNA flare probe comprises a fluorescent dye covalently attached to one or more nucleotides, and 28 nucleotides that are complementary to the nucleic acid sequence of the sequencing by synthesis primer-dimer.

66. The primer-dimer detection probe according to claim 62, wherein the LNA probe pair comprises:
a first LNA probe comprising a fluorescent dye and a series of nucleotides that are complementary to the nucleic acid sequence of the sequencing by synthesis adapter; and
a second LNA probe comprising a fluorescence quencher and a series of nucleotides that are complementary to the first LNA probe.

67. The primer-dimer detection probe according to any one of claims 60 to 66, wherein the fluorescent dye is a pyrene, a xanthene, a cyanine, an indole, a benzofuran, a coumarin, or a borapolyazaindacene, and is detectably distinct from an adapter-specific probe.

68. The primer-dimer detection probe according to any of claims 61 to 67, wherein the quencher is a BLACK HOLE QUENCHER® dye, an IOWA BLACK® Quencher, a QSY® Quencher, Dabsyl, Dabcel, a Deep Dark Quencher, and an ECLIPSE® Quencher.

69. A kit for detecting or quantifying nucleic acid, comprising:
one or more adapter-specific probe according to any one of claims 50 to 59;
a buffer; and
instructions for detecting or quantifying nucleic acid.

70. The kit of claim 69, further comprising a primer-dimer detection probe.

71. The kit of claim 69 or 70, wherein the adapter-specific probe comprises:
an oligonucleotide sequence that is complementary to a nucleic acid sequence of a sequencing by synthesis adapter, or a portion thereof; and
a fluorescent dye.

72. The kit according to any one of claims 69 to 71, wherein the adapter-specific probe further comprises a quencher.

73. The kit according to any one of claims 69 to 72, wherein the adapter-specific probe comprises a peptide nucleic acid (PNA) oligomer probe, a molecular beacon probe, a DNA flare probe or a locked nucleic acid (LNA) probe pair.

74. The kit according to claim 73, wherein the adapter-specific probe comprises a peptide nucleic acid (PNA) oligomer comprising a fluorescent dye, a quencher, and a series of nucleotides that are complementary to the nucleic acid sequence of the sequencing by synthesis adapter.

75. The kit according to claim 73, wherein the adapter-specific probe comprises a molecular beacon comprising a fluorescent dye, a quencher, and 18 nucleotides that are complementary to the nucleic acid sequence of the sequencing by synthesis adapter.

76. The kit according to claim 73, wherein the adapter-specific probe comprises a DNA flare probe comprising a fluorescent dye covalently attached to one or more nucleotides, and 28 nucleotides that are complementary to the nucleic acid sequence of the sequencing by synthesis adapter.

77. The kit according to claim 73, wherein the adapter-specific probe comprises a locked nucleic acid (LNA) probe pair comprising:
a first LNA probe comprising a fluorescent dye and a series of nucleotides that are complementary to the nucleic acid sequence of the sequencing by synthesis adapter; and
a second LNA probe comprising a fluorescence quencher and a series of nucleotides that are complementary to the first LNA probe.

78. The kit according to claim 70, wherein the primer-dimer detection probe comprises:
an oligonucleotide sequence that is complementary to a nucleic acid sequence of a sequencing by synthesis primer-dimer; and
a fluorescent dye.

79. The kit according to claim 78, wherein the primer-dimer detection probe further comprises a fluorescence quencher.

80. The kit according to claim 78, wherein the primer-dimer detection probe comprises a peptide nucleic acid (PNA) oligomer probe, a molecular beacon probe, a DNA flare probe or a locked nucleic acid (LNA) probe pair.

81. The kit according to claim 78, wherein the peptide nucleic acid (PNA) oligomer probe comprises a fluorescent dye, a quencher, and a series of nucleotides that are complementary to the nucleic acid sequence of the sequencing by synthesis primer-dimer.

82. The kit according to claim 78, wherein the molecular beacon probe comprises a fluorescent dye, a quencher, and 18 nucleotides that are complementary to the nucleic acid sequence of the sequencing by synthesis primer-dimer.

83. The kit according to claim 78, wherein the DNA flare probe comprises a fluorescent dye covalently attached to one or more nucleotides, and 28 nucleotides that are complementary to the nucleic acid sequence of the sequencing by synthesis primer-dimer.

84. The kit according to claim 78 wherein the locked nucleic acid (LNA) probe pair comprises:
a first LNA probe comprising a fluorescent dye and a series of nucleotides that are complementary to the nucleic acid sequence of the sequencing by synthesis primer-dimer; and
a second LNA probe comprising a fluorescence and a series of nucleotides that are complementary to the nucleic acid sequence of the sequencing by synthesis primer-dimer.

85. The kit according to any one of claims 70 to 84, wherein the primer-dimer detection probe has a detectable optical response that is distinguishable from the detectable optical response of the adapter-specific probe.


SEQUENCE LISTING (33 sequences)

SEQ ID NO: 1 - DNA, 63 bp, Artificial Sequence
  source: /note=“Description of Artificial Sequence Synthetic probe”
  source: /note=“5′ amine group”
  modified_base (4)..(4): Fluorescein-dT
  modified_base (16)..(16): Fluorescein-dT
  modified_base (28)..(28): Fluorescein-dT
  modified_base (40)..(40): Fluorescein-dT
  modified_base (52)..(52): Fluorescein-dT
gactgactgactgactgactgactgactgactgactgactgactgactgactgactgactgac

SEQ ID NO: 2 - DNA, 63 bp, Artificial Sequence
  source: /note=“Description of Artificial Sequence Synthetic probe”
gtcagtcagtcagtcagtcagtcagtcagtcagtcagtcagtcagtcagtcagtcagtcagtc

SEQ ID NO: 3 - DNA, 51 bp, Artificial Sequence
  source: /note=“Description of Artificial Sequence Synthetic probe”
  source: /note=“5′ amine group”
  modified_base (4)..(4): Fluorescein-dT
  modified_base (16)..(16): Fluorescein-dT
  modified_base (28)..(28): Fluorescein-dT
  modified_base (40)..(40): Fluorescein-dT
gactgactgactgactgactgactgactgactgactgactgactgactgac

SEQ ID NO: 4 - DNA, 51 bp, Artificial Sequence
  source: /note=“Description of Artificial Sequence Synthetic probe”
gtcagtcagtcagtcagtcagtcagtcagtcagtcagtcagtcagtcagtc

SEQ ID NO: 5 - DNA, 39 bp, Artificial Sequence
  source: /note=“Description of Artificial Sequence Synthetic probe”
  source: /note=“5′ amine group”
  modified_base (4)..(4): Fluorescein-dT
  modified_base (16)..(16): Fluorescein-dT
  modified_base (28)..(28): Fluorescein-dT
gactgactgactgactgactgactgactgactgactgac

SEQ ID NO: 6 - DNA, 39 bp, Artificial Sequence
  source: /note=“Description of Artificial Sequence Synthetic probe”
gtcagtcagtcagtcagtcagtcagtcagtcagtcagtc

SEQ ID NO: 7 - DNA, 27 bp, Artificial Sequence
  source: /note=“Description of Artificial Sequence Synthetic probe”
  source: /note=“5′ amine group”
  modified_base (4)..(4): Fluorescein-dT
  modified_base (16)..(16): Fluorescein-dT
gactgactgactgactgactgactgac

SEQ ID NO: 8 - DNA, 27 bp, Artificial Sequence
  source: /note=“Description of Artificial Sequence Synthetic probe”
gtcagtcagtcagtcagtcagtcagtc

SEQ ID NO: 9 - DNA, 15 bp, Artificial Sequence
  source: /note=“Description of Artificial Sequence Synthetic probe”
  source: /note=“5′ amine group”
  modified_base (4)..(4): Fluorescein-dT
gactgactgactgac

SEQ ID NO: 10 - DNA, 15 bp, Artificial Sequence
  source: /note=“Description of Artificial Sequence Synthetic probe”
gtcagtcagtcagtc

SEQ ID NO: 11 - DNA, 71 bp, Artificial Sequence
  source: /note=“Description of Artificial Sequence Synthetic probe”
  source: /note=“5′ amine group”
  modified_base (4)..(4): Fluorescein-dT
  modified_base (16)..(16): Fluorescein-dT
  modified_base (28)..(28): Fluorescein-dT
  modified_base (40)..(40): Fluorescein-dT
  modified_base (52)..(52): Fluorescein-dT
  modified_base (64)..(64): Fluorescein-dT
gactgactgactgactgactgactgactgactgactgactgactgactgactgactgactgactgactgac

SEQ ID NO: 12 - DNA, 71 bp, Artificial Sequence; identical to SEQ ID NO: 11
  source: /note=“Description of Artificial Sequence Synthetic probe”
  source: /note=“5′ amine group”
  modified_base (4)..(4): Fluorescein-dT
  modified_base (36)..(36): Fluorescein-dT
  modified_base (68)..(68): Fluorescein-dT

SEQ ID NO: 13 - DNA, 71 bp, Artificial Sequence; identical to SEQ ID NO: 11
  source: /note=“Description of Artificial Sequence Synthetic probe”
  source: /note=“5′ amine group”
  modified_base (4)..(4): Fluorescein-dT
  modified_base (20)..(20): Fluorescein-dT
  modified_base (36)..(36): Fluorescein-dT
  modified_base (52)..(52): Fluorescein-dT
  modified_base (68)..(68): Fluorescein-dT

SEQ ID NO: 14 - DNA, 71 bp, Artificial Sequence; identical to SEQ ID NO: 11
  source: /note=“Description of Artificial Sequence Synthetic probe”
  source: /note=“5′ amine group”
  modified_base (4)..(4): Fluorescein-dT
  modified_base (12)..(12): Fluorescein-dT
  modified_base (20)..(20): Fluorescein-dT
  modified_base (28)..(28): Fluorescein-dT
  modified_base (36)..(36): Fluorescein-dT
  modified_base (44)..(44): Fluorescein-dT
  modified_base (52)..(52): Fluorescein-dT
  modified_base (60)..(60): Fluorescein-dT
  modified_base (68)..(68): Fluorescein-dT

SEQ ID NO: 15 - DNA, 71 bp, Artificial Sequence
  source: /note=“Description of Artificial Sequence Synthetic probe”
gtcagtcagtcagtcagtcagtcagtcagtcagtcagtcagtcagtcagtcagtcagtcagtcagtcagtc

SEQ ID NO: 16 - DNA, 23 bp, Artificial Sequence
  source: /note=“Description of Artificial Sequence Synthetic probe”
  source: /note=“5′ amine group”
  modified_base (4)..(4): Fluorescein-dT
  modified_base (7)..(7): Fluorescein-dT
  modified_base (10)..(10): Fluorescein-dT
  modified_base (13)..(13): Fluorescein-dT
  modified_base (16)..(16): Fluorescein-dT
  modified_base (19)..(19): Fluorescein-dT
gactgattgtcttactgattgac

SEQ ID NO: 17 - DNA, 23 bp, Artificial Sequence
  source: /note=“Description of Artificial Sequence Synthetic probe”
gtcaatcagtaagacaatcagtc

SEQ ID NO: 18 - DNA, 28 bp, Artificial Sequence
  source: /note=“Description of Artificial Sequence Synthetic probe”
  source: /note=“5′ amine group”
  modified_base (4)..(4): Fluorescein-dT
  modified_base (8)..(8): Fluorescein-dT
  modified_base (12)..(12): Fluorescein-dT
  modified_base (16)..(16): Fluorescein-dT
  modified_base (20)..(20): Fluorescein-dT
  modified_base (24)..(24): Fluorescein-dT
gactgactgactgactgactgactgact

SEQ ID NO: 19 - DNA, 28 bp, Artificial Sequence
  source: /note=“Description of Artificial Sequence Synthetic probe”
agtcagtcagtcagtcagtcagtcagtc

SEQ ID NO: 20 - DNA, 33 bp, Artificial Sequence
  source: /note=“Description of Artificial Sequence Synthetic probe”
  source: /note=“5′ amine group”
  modified_base (4)..(4): Fluorescein-dT
  modified_base (9)..(9): Fluorescein-dT
  modified_base (14)..(14): Fluorescein-dT
  modified_base (19)..(19): Fluorescein-dT
  modified_base (24)..(24): Fluorescein-dT
  modified_base (29)..(29): Fluorescein-dT
gactgacttactgtctgattgactgacttactg

SEQ ID NO: 21 - DNA, 33 bp, Artificial Sequence
  source: /note=“Description of Artificial Sequence Synthetic probe”
cagtaagtcagtcaatcagacagtaagtcagtc

SEQ ID NO: 22 - DNA, 38 bp, Artificial Sequence
  source: /note=“Description of Artificial Sequence Synthetic probe”
  source: /note=“5′ amine group”
  modified_base (4)..(4): Fluorescein-dT
  modified_base (10)..(10): Fluorescein-dT
  modified_base (16)..(16): Fluorescein-dT
  modified_base (22)..(22): Fluorescein-dT
  modified_base (28)..(28): Fluorescein-dT
  modified_base (34)..(34): Fluorescein-dT
gactgatcgtctgactgactgtctgactgactgtctga

SEQ ID NO: 23 - DNA, 38 bp, Artificial Sequence
  source: /note=“Description of Artificial Sequence Synthetic probe”
tcagacagtcagtcagacagtcagtcagacgatcagtc

SEQ ID NO: 24 - DNA, 43 bp, Artificial Sequence
  source: /note=“Description of Artificial Sequence Synthetic probe”
  source: /note=“5′ amine group”
  modified_base (4)..(4): Fluorescein-dT
  modified_base (11)..(11): Fluorescein-dT
  modified_base (18)..(18): Fluorescein-dT
  modified_base (25)..(25): Fluorescein-dT
  modified_base (32)..(32): Fluorescein-dT
  modified_base (39)..(39): Fluorescein-dT
gactgactgattgactgtctgacttactgactgactgattgac

SEQ ID NO: 25 - DNA, 43 bp, Artificial Sequence
  source: /note=“Description of Artificial Sequence Synthetic probe”
gtcaatcagtcagtcagtaagtcagacagtcaatcagtcagtc

SEQ ID NO: 26 - DNA, 48 bp, Artificial Sequence
  source: /note=“Description of Artificial Sequence Synthetic probe”
  source: /note=“5′ amine group”
  modified_base (4)..(4): Fluorescein-dT
  modified_base (12)..(12): Fluorescein-dT
  modified_base (20)..(20): Fluorescein-dT
  modified_base (28)..(28): Fluorescein-dT
  modified_base (36)..(36): Fluorescein-dT
  modified_base (44)..(44): Fluorescein-dT
gactgactgactgactgactgactgactgactgactgactgactgact

SEQ ID NO: 27 - DNA, 48 bp, Artificial Sequence
  source: /note=“Description of Artificial Sequence Synthetic probe”
agtcagtcagtcagtcagtcagtcagtcagtcagtcagtcagtcagtc

SEQ ID NO: 28 - DNA, 53 bp, Artificial Sequence
  source: /note=“Description of Artificial Sequence Synthetic probe”
  source: /note=“5′ amine group”
  modified_base (4)..(4): Fluorescein-dT
  modified_base (13)..(13): Fluorescein-dT
  modified_base (22)..(22): Fluorescein-dT
  modified_base (31)..(31): Fluorescein-dT
  modified_base (40)..(40): Fluorescein-dT
  modified_base (49)..(49): Fluorescein-dT
gactgactgacttactgactgtctgactgattgactgactgactgacttactg

SEQ ID NO: 29 - DNA, 53 bp, Artificial Sequence
  source: /note=“Description of Artificial Sequence Synthetic probe”
cagtaagtcagtcagtcagtcaatcagtcagacagtcagtaagtcagtcagtc

SEQ ID NO: 30 - DNA, 58 bp, Artificial Sequence
  source: /note=“Description of Artificial Sequence Synthetic probe”
  source: /note=“5′ amine group”
  modified_base (4)..(4): Fluorescein-dT
  modified_base (14)..(14): Fluorescein-dT
  modified_base (24)..(24): Fluorescein-dT
  modified_base (34)..(34): Fluorescein-dT
  modified_base (44)..(44): Fluorescein-dT
  modified_base (54)..(54): Fluorescein-dT
gactgactgactgtctgactgactgactgactgtctgactgactgactgactgtctga

SEQ ID NO: 31 - DNA, 58 bp, Artificial Sequence
  source: /note=“Description of Artificial Sequence Synthetic probe”
tcagacagtcagtcagtcagtcagacagtcagtcagtcagtcagacagtcagtcagtc

SEQ ID NO: 32 - DNA, 63 bp, Artificial Sequence
  source: /note=“Description of Artificial Sequence Synthetic probe”
  source: /note=“5′ amine group”
  modified_base (4)..(4): Fluorescein-dT
  modified_base (15)..(15): Fluorescein-dT
  modified_base (26)..(26): Fluorescein-dT
  modified_base (37)..(37): Fluorescein-dT
  modified_base (48)..(48): Fluorescein-dT
  modified_base (59)..(59): Fluorescein-dT
gactgactgactgattgactgactgtctgactgacttactgactgactgactgactgattgac

SEQ ID NO: 33 - DNA, 63 bp, Artificial Sequence
  source: /note=“Description of Artificial Sequence Synthetic probe”
gtcaatcagtcagtcagtcagtcagtaagtcagtcagacagtcagtcaatcagtcagtcagtc